SEQUENCE LISTING (112 sequences)

SEQ ID NO: 1 - DNA, 403 bp, Cannabis sp.
  source 1..403: mol_type = genomic DNA
  variation 202: allele = N is T or C
ccattacaaaacttgaattatataaagaaaaatgtgaatcaataaaattacaaaagagagaaaaaaaaaagtatataagagaaagaaaaaccatatcctcctcaaaagggtatgtatatttttctgcaaatttgacattgtaacaactactcttgattttctcaatatatttttaattttttttttatctttgactattatnaattgcagtagttagaggtggaatagcattgtttggtgttgattgaattggaaaattaagacgccaaagcatgatcttagagcacccatctggtgggataccttttatttcagtttcatcttcaagaaccacttgcttcacaaatttgattgaatcctgctcaaaaatatattaatttaataataattaagtacagctaat

SEQ ID NO: 2 - DNA, 403 bp, Cannabis sp.
  source 1..403: mol_type = genomic DNA
  variation 202: allele = N is C or T
ctagtacaacgattcagctaaaaactcaggaaactcatcaagcaacatctcgttctctcgagctagcaaggcttccacaaatgaagtattatccatatctttagatccttcactctgattttggcttagatcattctgtgaagaagaataattcatcaactcagccattgatgtctcatgttcttgagcttgatcatttggnaaaaatgcatcttcaagaagaaagtcattccaattgaagctggctacaccagctagaggtggcatctcttgggtactggatgaagatgatgatgagggtgaagaaaatgttgcttctgggtagaaacaaggcaataatgaagattcataattattgggtttgtagtttgaagcttcagtactgaccaactttatggcttgg

SEQ ID NO: 3 - DNA, 403 bp, Cannabis sp.
  source 1..403: mol_type = genomic DNA
  variation 202: allele = N is A or T
ggagatgatcaagaaaggaaaacatcctaatgttgtgacatatgcaatgataatggaaggtttgtgtttgttgggaaagtattcggaagcaaagaagatgatgtttgatatggattatcgagggtgtaaaccaaagcttgtgaactttggtattttgatgactgatcttggaaaaagaggtaagattgaggaggcaagagcnttagttagtgagatgaagaaaaggaagtttaaacctgatgttgtgagctataatatattgataaattatctgtgcaaggaaggtaaggcaatggaggcatataaaattttgatggaaatgcaagttgaaggttgtgaaccaaatgcagctacatacaggatgatggttgatgggttttgcagggtgggcgatttcgaaggt

SEQ ID NO: 4 - DNA, 403 bp, Cannabis sp.
  source 1..403: mol_type = genomic DNA
  variation 202: allele = N is A or G
cggaagtaaccacttcactaattgcaatatggtatacttagcttgatacatagttttcttgttacatcttaatccattctgattatttttctgctgtctttgcaactgatgaatagtttcttggctatgtatcttcagtgtaaacacagaagccataacattcaactttacgtatggccttagtgctgcagccaggtatggnataaatcaaacttacactaatctttcaaattcaacatttcatttatctcatttttggtattgttgttacaacagcacaagggtatcgaatgagttaggggcagggcgtccggacagagctaagagtgcaatgattgttaccctgaagctatgtggagttcttgccttgatactagttttggctctaggattcggacacaac

SEQ ID NO: 5 - DNA, 403 bp, Cannabis sp.
  source 1..403: mol_type = genomic DNA
  variation 202: allele = N is C or A
ttttatccaatccaatcctatatcaaaccccactacccaaacgtaaccaacgtccacccaaaaaaaaaagcaaatggagctgcaatccgaacaggtataatatcataatgctagctcggttagtgaaaaacaccacattttaaaagaactgcatgtatttcaaaagcagtattaccttccaactgtaggaaacatttttccngctggctgaagaaaccgatctcttgcaattacataggactccagcattctttcattgactaataaggttcctaaagacacaaagagaaattatatatgaactcactccagcaaaaatagagcttacaatacccataacccactttaaatcaacactgttgataattttttattctagtttcctttttctggttttccttat

SEQ ID NO: 6 - DNA, 403 bp, Cannabis sp.
  source 1..403: mol_type = genomic DNA
  variation 202: allele = N is A or C
aaacgttaccaaaccagaagagaccaacattgctacattctcgattaccagtgctacaaaccaaccgacgatcgaaagctcaacaccgagttttgtggcgaactcattaggcggaccaagaatctaggcctcatcgagtacaagtttctcctcaaggctattgtcagtgctggaatcggtgaacagacttacgctccgagantcatcttcgacggtcgtgaagattctcctactattgctgatggaatctctgagatggaggagtttttctttgacagcgttggaagacttctcagacgtaacggaatatctccgtctcagatcgatgttctcgttgttaacgtttcgatgctttcgactgttccttctttgtcttctcggattataaatcattacaagatga

SEQ ID NO: 7 - DNA, 403 bp, Cannabis sp.
  source 1..403: mol_type = genomic DNA
  variation 202: allele = N is T or C
aaaaaatatggatgaaaaataaaataaacatcacagctttttgtaccccctctctctctaaagacgtacgtactaataataataataattaactacaaagtttggataattaaaacaagttgttttataagttttgatgatgagaagatagtatatataatatattggatcatatcaaatcaactaaacagctagctgttgntgtgtttgatgagatgattgatcttgatcttcattgaagcacaagtgaagatttagtgattttccagaagtaataagagaagaagtagttagttgtagtggtggttgcattagaagcatttctctttggagataacaacaataggtttggaatgtggttggagtcacattcaactgaaaccctaccccaaataggaaatcc

SEQ ID NO: 8 - DNA, 403 bp, Cannabis sp.
  source 1..403: mol_type = genomic DNA
  variation 202: allele = N is T or C
tcatcatcatcatcataaccctcatcatcaccctccaacctctctgaatcagatcaatctaccaccctgcacacctcaagaatttcatggttcttttctctgtttcacttattgcctcttctctgtcttctttcattctttttttagattattgagatactataattgtttcatggtggcaggagtggcatcgtttctaggnaagagatcggtatcgttttcgggtatcgagctaggagaagaaggcaatggaggagaagatgatttatccgatgatggatctcaagcaggggaaaagaagaggagacttaatatggaacaggttaagacccttgagaagaactttgaattggggaacaagcttgagccagaaaggaaaatgcagctagctagagctcttggt

SEQ ID NO: 9 - DNA, 403 bp, Cannabis sp.
  source 1..403: mol_type = genomic DNA
  variation 202: allele = N is G or A
tatattgttttggttacagttggaattgaggcagaggatgagaaggttaacttccttttgactgaggtcaagggcaaagatctcactgagctaattgcttccggaagggagaagctagcatcagttccgtcaggtggtggtggtggtgcagttgcttactctgcaccatcaggtggagcaggcgccgccccagctgctgctnccgagtcaaagaaggaagagaaagtagaagagaaagaagagtcagatgatgtaagtttcatatagttgttaagtctttaaagtctctggttttggctgtttttaagtcattgaatttgctctaatggtatgctgtttattctttgtttcaggatatgggtttcagtctcttcgactaaaaatctttagcttttatcaggaa

SEQ ID NO: 10 - DNA, 403 bp, Cannabis sp.
  source 1..403: mol_type = genomic DNA
  variation 202: allele = N is G or A
gtgagggatttttccattgagaccctgaatttagtgtagtgctcgtacaaaccaccatccaatgccactacagacttctgtttatctcccaattttactgtgtctcttcccaatttcttgaggattcctaagatccctgcggctgatagtcgagctcctcgagtggcgacaatatcacaaatctccactattgtttttctcnttttgagggaggtgttagatatctgacaaagagagatggacaacttattataaccatcaaacgtggaaataaactggtactaattaaggaaaataatatattctatgatgtttagtagggtcctaatgtttagttcaactgcagagaaagtaatcaactcaaaggttgaactgtccctaactagttagtctttcctcacct

SEQ ID NO: 11 - DNA, 403 bp, Cannabis sp.
  source 1..403: mol_type = genomic DNA
  variation 202: allele = N is G or T
gttcgatgaagtcaatacccgaacccctataatccttgcaaaactcgaaatttgaaatgctaagaaaggatcatgtaaggtgcgttcttgacttctccaaaatatttgcatttcctcaacaccttagatttctttatcttacttctttttctttcccctgctgcttttcactaatttcgaccatcttcttctatcaaatatnactgcaaaaaacactaatcaaaaccccaattaatcacaactgagaaggcaatcggattattgcattgttataactaattatagtactacattacacttgcactatatattttgtgctccaaaactcatgaatgaaataatcacgtgccattgtttttggcaatatattgagtttgtaagaattatagaaaatctctattaa

SEQ ID NO: 12 - DNA, 403 bp, Cannabis sp.
  source 1..403: mol_type = genomic DNA
  variation 202: allele = N is A or G
tggatctcattaaagtgactagatatttgatactaatggtcaagttcactcttgacttgtacatatttccatgatcaaaactaccctaataaagttgctcattgtgacttatgaggtgtgctagcgtcattgcagactctttaatcatagattgcacttatatataaagttgctttaaataaagagtcgtgagcttcaactnaaaacctcaaagatatctcattttatcatttattctttcttagaattttttaacaccctttgaactatatataatggaagttgagaggactccagctactaaagaaagcaaatatgaggtaagtttcaccaagaaacatattgtgaaagcattaaatatttcctcattaccaaaatcaagcatcttaaccctttccaattt

SEQ ID NO: 13 - DNA, 403 bp, Cannabis sp.
  source 1..403: mol_type = genomic DNA
  variation 202: allele = N is C or T
cacaaactcggccacaaattcaagtagttctggcttaaacatctgattccaaagtaccaaagcccactcacttggctggttaagcccataggcttcagcaactattagtgcctcttgaaaacgggactgctcaactaagactctcctggcatttgtctctgacaggtaaagccattgaatgtcaggcatccggatttggagngacagaagggaagcctgagcacaagctctacgtgctttgttgccagcatcaagggaggagtgaacttcagcagcttcaatgaaatagcgcatagcatctaataggtcttcattctggtcattgtctttacgaccaaaccactgcccagaggactgatctgctcgagattctaaaagagcagctgtttcatgtttcatgtca

SEQ ID NO: 14 - DNA, 403 bp, Cannabis sp.
  source 1..403: mol_type = genomic DNA
  variation 202: allele = N is A or G
gcccattcatacctttatggttgtggctgcatagtccacacagccatatgtattgcagtccactggagcaacatttaagaacattctcagagttttaacacataacttccaacaataaactagcattcctgtgacacttgttacttatctaagtttgctataacaagattatgtgctacattttggtaatgtttaagcatcnaattgatccaattcgaaaaacttgaagtagatggaaaattgatgctacattggtagctgctagttattatcacaaagattaatatttggaatatgtaaatgtacgtatgaacaatgaactgtaatttcagtcttcttatatttaacaagaagtaatcaagttagggtattccaaaaacagtgttaaaattgagtttctttg

SEQ ID NO: 15 - DNA, 403 bp, Cannabis sp.
  source 1..403: mol_type = genomic DNA
  variation 202: allele = N is T or G
tcccgttacggcccagcagccaccgtcttcaacggcccagttaggaagtggaagaagaaatgggtccatgtttcctcctcctcttcttcttcaaccctcaacgcctacaaccacaaccctcaatcacaatccaacggcaccccaaccccccgcctcctcctctgccgatggactcccgccaccgctacttcagccgccgctnccgacggctccggcggaacgcagttggaagagccaccgaggaggaagttccggtatactcctgtaagtgaaaatcacttgattatgctttagggtttatgggtattattggttctgctgttacatatatgggtttgttggcgtgtctgtgtttggttagattatgcatatgaatatgaaatatggatttgttttcttttct

SEQ ID NO: 16 - DNA, 403 bp, Cannabis sp.
  source 1..403: mol_type = genomic DNA
  variation 202: allele = N is C or A
aagttaacaggtatgtttcagattttgtcttactttcagcattttcttcgctttttcaagctcaaaaggatcagggtgatttgcactgaaaacagtctccacctgaaaaataaatgagtgtaactaagtaaacagaaaacaacataccaacaaatttggctgaaatacatttatcatacatacctccttgaccaatgcatcngtgttaagtaattcaatatcatctgatactttttttgcaatcccattttgtgttgggggaaaatctttctttggctgcaccttacttggtcctctccctcttccagaacctggaatggcaccaccacgattcgagggtttcttgtggccacgaccatggccaccgtggtttcctccatgcgatataaccaaatcagcacca

SEQ ID NO: 17 - DNA, 403 bp, Cannabis sp.
  source 1..403: mol_type = genomic DNA
  variation 202: allele = N is A or G
aacttaaaaggaaaagaattgaacttgaaactctgtatagcatcatggctgttttctttgtcttctcttctctttgaattcaataggtacttgatctgtgagaagctggcactgtttttattggttcataccaattttaagatattcctttttattttattttttggtttacgtaatatttaatttgaacatgtaacagaanttgggctttgcatcctgtaagcggaacttggaagctacagaaggaattaatgttcttattgagaagaaaaataatgatgggaagtgggcctatggtttatcatgcattgaatatactgagtttgaaaagtttgggattgcagatggacatcattcaaccaataggtatagtctggttcacttacaaagttggcataatcaa

SEQ ID NO: 18 - DNA, 403 bp, Cannabis sp.
  source 1..403: mol_type = genomic DNA
  variation 202: allele = N is C or T
atttaactctccggttatgtcgacaaagccccgagctgttcttgacagcgctagcaaaatgcgagcgtcttatggattgaagcaggggcagtcccgtctttttcacgagctcccatctgggctgaatatggagttgattgtacagaagggtgctgtagataataaagacccagatgataaatgccagacaagaattgataanccatctctggtttttgttcatggaagctatcatgctgcttggtgctgggctgaacactggttacccttcttttcatcacatggctacgattgctatgctcttagcttgttgggccaggttctttctctcatcttaccttctccttttcatcaaggctgtcaaatccgtttcttttcatctttaatgaatcgttaaatgatt

SEQ ID NO: 19 - DNA, 403 bp, Cannabis sp.
  source 1..403: mol_type = genomic DNA
  variation 202: allele = N is C or G
ggcaaagcaaaagcaaattaagacaaacccacatatcacaattcacaactcaaaactcaaaactcacaagttattatatattactccattcctttgtttctgagaaccattagagaaaagaatgggtgccgttgtgttaaaccagagtgtacacataaatcccagcaaagggcctcaaaaattaattaacaacgatgtgatngaagaaattggagggcttattaaagtgtacaaagacggaaaagtcgaaagaccagaggtagtgccatgtgtcactgcttcattggctgatcacgatcaactgggatcagcagcagtgatttctacggacgtggtcattgacaagtccaccaatgtttgggctcgtttttatgttccaattttggctcagaaagacaaaatc

SEQ ID NO: 20 - DNA, 403 bp, Cannabis sp.
  source 1..403: mol_type = genomic DNA
  variation 202: allele = N is G or A
tgcagctcactgccttaaaccgcatcttttcaacatatagatctgcagaaattttaatactgtgttatataatatgatcattcttttaaaaaaaaaatcattaaaatggagatagcaacttggctaaaactcacccattacacaagtgttcaaatttggagctgttttaaagagcctgaagaacataacatagtttccagangtaacagcagcacgaactgcaagagcatgctttacagcattatccctttttgcttcccttgataatctgaactcaatttcacaacaatattgaaggtcattacaagagtttcaacatagtagaaggatggtaaatagctaatgaaatttcctcaaatcataactgaccttgacatggatgatacaagatctctgttgttac

SEQ ID NO: 21 - DNA, 403 bp, Cannabis sp.
  source 1..403: mol_type = genomic DNA
  variation 202: allele = N is C or A
tgttactgattctctcataagatagagaagagaaaaaaaaaaaaggggaggagggaagattttcagaatatatgttcttcccttactttgcatcaaacccagcttttattatatatatgattaataatatatatatttatatatatttcttctgtccatattaattttcttccttaagcccatccaaaaccaaatcctctantgaagcttatattcccttttgtcagcaagagtccatttatcttgacccacgtttcaagcattaaattttgtgagaatacaagttttgataccattccctgtccaaagggtactctctagctaataatcactatatatccaaattaattcaaaccatattcacattaagaaataaccaaagttagctctctctttctctcat

SEQ ID NO: 22 - DNA, 403 bp, Cannabis sp.
  source 1..403: mol_type = genomic DNA
  variation 202: allele = N is A or G
agatgataactgttgtggtggacaaccagacggtggtatggatgaacttttagctgttttgggttataaagttaggtcatctgacatggctgaagttgctcaaaagcttgaacagcttgaagaagctatgtgtagtgttcaacaagataatctttcacaacttgcttctgatactgttcattataatccttctgatttatcnacatggttggaaagtatgcttacagagcttaatccttctcctcctaattttgattctgtaatggtaccaccaccaccaccaccaccacaatcacaacctcaaaggccttcgattattgaagaaacttcttacttagctccagctgaatcttcaaccataacttctattgatttcccagatcagagaaatcaaaactcgtta

SEQ ID NO: 23 - DNA, 22 bp, synthetic construct
  source 1..22: mol_type = other DNA
accatatcctcctcaaaagggt

SEQ ID NO: 24 - DNA, 23 bp, synthetic construct
  source 1..23: mol_type = other DNA
tcaaatttgtgaagcaagtggtt

SEQ ID NO: 25 - DNA, 22 bp, synthetic construct
  source 1..22: mol_type = other DNA
tcaaatttgtgaagcaagtggt

SEQ ID NO: 26 - DNA, 23 bp, synthetic construct
  source 1..23: mol_type = other DNA
aaccatatcctcctcaaaagggt

SEQ ID NO: 27 - DNA, 20 bp, synthetic construct
  source 1..20: mol_type = other DNA
tctctcgagctagcaaggct

SEQ ID NO: 28 - DNA, 21 bp, synthetic construct
  source 1..21: mol_type = other DNA
gccttgtttctacccagaagc

SEQ ID NO: 29 - DNA, 20 bp, synthetic construct
  source 1..20: mol_type = other DNA
ttctctcgagctagcaaggc

SEQ ID NO: 30 - DNA, 20 bp, synthetic construct
  source 1..20: mol_type = other DNA
gagctagcaaggcttccaca

SEQ ID NO: 31 - DNA, 21 bp, synthetic construct
  source 1..21: mol_type = other DNA
tcgagggtgtaaaccaaagct

SEQ ID NO: 32 - DNA, 19 bp, synthetic construct
  source 1..19: mol_type = other DNA
cttcgaaatcgcccaccct

SEQ ID NO: 33 - DNA, 19 bp, synthetic construct
  source 1..19: mol_type = other DNA
accttcgaaatcgcccacc

SEQ ID NO: 34 - DNA, 20 bp, synthetic construct
  source 1..20: mol_type = other DNA
ccctgcaaaacccatcaacc

SEQ ID NO: 35 - DNA, 20 bp, synthetic construct
  source 1..20: mol_type = other DNA
acgtatggccttagtgctgc

SEQ ID NO: 36 - DNA, 19 bp, synthetic construct
  source 1..19: mol_type = other DNA
tgtgtccgaatcctagagc

SEQ ID NO: 37 - DNA, 20 bp, synthetic construct
  source 1..20: mol_type = other DNA
cgtatggccttagtgctgca

SEQ ID NO: 38 - DNA, 20 bp, synthetic construct
  source 1..20: mol_type = other DNA
gtgtccgaatcctagagcca

SEQ ID NO: 39 - DNA, 20 bp, synthetic construct
  source 1..20: mol_type = other DNA
aaaccccactacccaaacgt

SEQ ID NO: 40 - DNA, 24 bp, synthetic construct
  source 1..24: mol_type = other DNA
gctctatttttgctggagtgagtt

SEQ ID NO: 41 - DNA, 22 bp, synthetic construct
  source 1..22: mol_type = other DNA
gctctatttttgctggagtgag

SEQ ID NO: 42 - DNA, 23 bp, synthetic construct
  source 1..23: mol_type = other DNA
gctctatttttgctggagtgagt

SEQ ID NO: 43 - DNA, 20 bp, synthetic construct
  source 1..20: mol_type = other DNA
gtgctacaaaccaaccgacg

SEQ ID NO: 44 - DNA, 20 bp, synthetic construct
  source 1..20: mol_type = other DNA
acatcgatctgagacggaga

SEQ ID NO: 45 - DNA, 20 bp, synthetic construct
  source 1..20: mol_type = other DNA
tgctacaaaccaaccgacga

SEQ ID NO: 46 - DNA, 20 bp, synthetic construct
  source 1..20: mol_type = other DNA
tcgaaagctcaacaccgagt

SEQ ID NO: 47 - DNA, 20 bp, synthetic construct
  source 1..20: mol_type = other DNA
agaaggaacagtcgaaagca

SEQ ID NO: 48 - DNA, 21 bp, synthetic construct
  source 1..21: mol_type = other DNA
acagctttttgtaccccctct

SEQ ID NO: 49 - DNA, 21 bp, synthetic construct
  source 1..21: mol_type = other DNA
tgcttctaatgcaaccaccac

SEQ ID NO: 50 - DNA, 21 bp, synthetic construct
  source 1..21: mol_type = other DNA
tcacagctttttgtaccccct

SEQ ID NO: 51 - DNA, 21 bp, synthetic construct
  source 1..21: mol_type = other DNA
gcttctaatgcaaccaccact

SEQ ID NO: 52 - DNA, 20 bp, synthetic construct
  source 1..20: mol_type = other DNA
accctcatcatcaccctcca

SEQ ID NO: 53 - DNA, 20 bp, synthetic construct
  source 1..20: mol_type = other DNA
cccctgcttgagatccatca

SEQ ID NO: 54 - DNA, 21 bp, synthetic construct
  source 1..21: mol_type = other DNA
tctcctcttcttttcccctgc

SEQ ID NO: 55 - DNA, 21 bp, synthetic construct
  source 1..21: mol_type = other DNA
ctcctcttcttttcccctgct

SEQ ID NO: 56 - DNA, 20 bp, synthetic construct
  source 1..20: mol_type = other DNA
cggaagggagaagctagcat

SEQ ID NO: 57 - DNA, 20 bp, synthetic construct
  source 1..20: mol_type = other DNA
gtcgaagagactgaaaccca

SEQ ID NO: 58 - DNA, 20 bp, synthetic construct
  source 1..20: mol_type = other DNA
gctaattgcttccggaaggg

SEQ ID NO: 59 - DNA, 20 bp, synthetic construct
  source 1..20: mol_type = other DNA
agcatcagttccgtcaggtg

SEQ ID NO: 60 - DNA, 20 bp, synthetic construct
  source 1..20: mol_type = other DNA
gatccctgcggctgatagtc

SEQ ID NO: 61 - DNA, 22 bp, synthetic construct
  source 1..22: mol_type = other DNA
agggacagttcaacctttgagt

SEQ ID NO: 62 - DNA, 20 bp, synthetic construct
  source 1..20: mol_type = other DNA
ttcctaagatccctgcggct

SEQ ID NO: 63 - DNA, 20 bp, synthetic construct
  source 1..20: mol_type = other DNA
attcctaagatccctgcggc

SEQ ID NO: 64 - DNA, 20 bp, synthetic construct
  source 1..20: mol_type = other DNA
ggtgcgttcttgacttctcc

SEQ ID NO: 65 - DNA, 20 bp, synthetic construct
  source 1..20: mol_type = other DNA
ccaaaaacaatggcacgtga

SEQ ID NO: 66 - DNA, 21 bp, synthetic construct
  source 1..21: mol_type = other DNA
ggtgcgttcttgacttctcca

SEQ ID NO: 67 - DNA, 21 bp, synthetic construct
  source 1..21: mol_type = other DNA
aggtgcgttcttgacttctcc

SEQ ID NO: 68 - DNA, 20 bp, synthetic construct
  source 1..20: mol_type = other DNA
gtgtgctagcgtcattgcag

SEQ ID NO: 69 - DNA, 22 bp, synthetic construct
  source 1..22: mol_type = other DNA
tggaaagggttaagatgcttga

SEQ ID NO: 70 - DNA, 20 bp, synthetic construct
  source 1..20: mol_type = other DNA
tgtgctagcgtcattgcaga

SEQ ID NO: 71 - DNA, 23 bp, synthetic construct
  source 1..23: mol_type = other DNA
ttggaaagggttaagatgcttga

SEQ ID NO: 72 - DNA, 20 bp, synthetic construct
  source 1..20: mol_type = other DNA
cccactcacttggctggtta

SEQ ID NO: 73 - DNA, 20 bp, synthetic construct
  source 1..20: mol_type = other DNA
gatcagtcctctgggcagtg

SEQ ID NO: 74 - DNA, 20 bp, synthetic construct
  source 1..20: mol_type = other DNA
agatcagtcctctgggcagt

SEQ ID NO: 75 - DNA, 20 bp, synthetic construct
  source 1..20: mol_type = other DNA
cagatcagtcctctgggcag

SEQ ID NO: 76 - DNA, 20 bp, synthetic construct
  source 1..20: mol_type = other DNA
tgcatagtccacacagccat

SEQ ID NO: 77 - DNA, 25 bp, synthetic construct
  source 1..25: mol_type = other DNA
acagttcattgttcatacgtacatt

SEQ ID NO: 78 - DNA, 20 bp, synthetic construct
  source 1..20: mol_type = other DNA
ctgcatagtccacacagcca

SEQ ID NO: 79 - DNA, 26 bp, synthetic construct
  source 1..26: mol_type = other DNA
acagttcattgttcatacgtacattt

SEQ ID NO: 80 - DNA, 20 bp, synthetic construct
  source 1..20: mol_type = other DNA
gggtccatgtttcctcctcc

SEQ ID NO: 81 - DNA, 20 bp, synthetic construct
  source 1..20: mol_type = other DNA
ccaaacacagacacgccaac

SEQ ID NO: 82 - DNA, 20 bp, synthetic construct
  source 1..20: mol_type = other DNA
cacagacacgccaacaaacc

SEQ ID NO: 83 - DNA, 20 bp, synthetic construct
  source 1..20: mol_type = other DNA
caaacacagacacgccaaca

SEQ ID NO: 84 - DNA, 22 bp, synthetic construct
  source 1..22: mol_type = other DNA
aaggatcagggtgatttgcact

SEQ ID NO: 85 - DNA, 20 bp, synthetic construct
  source 1..20: mol_type = other DNA
atggtcgtggccacaagaaa

SEQ ID NO: 86 - DNA, 21 bp, synthetic construct
  source 1..21: mol_type = other DNA
aggatcagggtgatttgcact

SEQ ID NO: 87 - DNA, 21 bp, synthetic construct
  source 1..21: mol_type = other DNA
ggatcagggtgatttgcactg

SEQ ID NO: 88 - DNA, 20 bp, synthetic construct
  source 1..20: mol_type = other DNA
tctgtgagaagctggcactg

SEQ ID NO: 89 - DNA, 21 bp, synthetic construct
  source 1..21: mol_type = other DNA
tgatgtccatctgcaatccca

SEQ ID NO: 90 - DNA, 20 bp, synthetic construct
  source 1..20: mol_type = other DNA
tgtgagaagctggcactgtt

SEQ ID NO: 91 - DNA, 22 bp, synthetic construct
  source 1..22: mol_type = other DNA
tgccaactttgtaagtgaacca

SEQ ID NO: 92 - DNA, 23 bp, synthetic construct
  source 1..23: mol_type = other DNA
tgccaactttgtaagtgaaccag

SEQ ID NO: 93 - DNA, 19 bp, synthetic construct
  source 1..19: mol_type = other DNA
ccccgagctgttcttgaca

SEQ ID NO: 94 - DNA, 20 bp, synthetic construct
  source 1..20: mol_type = other DNA
aacctggcccaacaagctaa

SEQ ID NO: 95 - DNA, 20 bp, synthetic construct
  source 1..20: mol_type = other DNA
cctggcccaacaagctaaga

SEQ ID NO: 96 - DNA, 20 bp, synthetic construct
  source 1..20: mol_type = other DNA
gaacctggcccaacaagcta

SEQ ID NO: 97 - DNA, 20 bp, synthetic construct
  source 1..20: mol_type = other DNA
agaatgggtgccgttgtgtt

SEQ ID NO: 98 - DNA, 20 bp, synthetic construct
  source 1..20: mol_type = other DNA
ttgtcaatgaccacgtccgt

SEQ ID NO: 99 - DNA, 20 bp, synthetic construct
  source 1..20: mol_type = other DNA
aagaatgggtgccgttgtgt

SEQ ID NO: 100 - DNA, 20 bp, synthetic construct
  source 1..20: mol_type = other DNA
gggtgccgttgtgttaaacc

SEQ ID NO: 101 - DNA, 20 bp, synthetic construct
  source 1..20: mol_type = other DNA
agctcactgccttaaaccgc

SEQ ID NO: 102 - DNA, 21 bp, synthetic construct
  source 1..21: mol_type = other DNA
tgctgtaaagcatgctcttgc

SEQ ID NO: 103 - DNA, 20 bp, synthetic construct
  source 1..20: mol_type = other DNA
cagctcactgccttaaaccg

SEQ ID NO: 104 - DNA, 20 bp, synthetic construct
  source 1..20: mol_type = other DNA
tcgtgctgctgttacctctg

SEQ ID NO: 105 - DNA, 20 bp, synthetic construct
  source 1..20: mol_type = other DNA
aaaaaggggaggagggaaga

SEQ ID NO: 106 - DNA, 21 bp, synthetic construct
  source 1..21: mol_type = other DNA
agagagtaccctttggacagg

SEQ ID NO: 107 - DNA, 22 bp, synthetic construct
  source 1..22: mol_type = other DNA
ggggaggagggaagattttcag

SEQ ID NO: 108 - DNA, 21 bp, synthetic construct
  source 1..21: mol_type = other DNA
ggggaggagggaagattttca

SEQ ID NO: 109 - DNA, 21 bp, synthetic construct
  source 1..21: mol_type = other DNA
agacggtggtatggatgaact

SEQ ID NO: 110 - DNA, 20 bp, synthetic construct
  source 1..20: mol_type = other DNA
tcgaaggcctttgaggttgt

SEQ ID NO: 111 - DNA, 22 bp, synthetic construct
  source 1..22: mol_type = other DNA
cagacggtggtatggatgaact

SEQ ID NO: 112 - DNA, 20 bp, synthetic construct
  source 1..20: mol_type = other DNA
cgaaggcctttgaggttgtg

(19) United States
(12) Patent Application Publication
(10) Pub. No.: US 2026/0144200 A1
(43) Pub. Date: May 28, 2026

(54) QUANTITATIVE TRAIT LOCI ASSOCIATED WITH FLOWERING TIME IN CANNABIS

(71) Applicant: Puregene AG, Zeiningen (CH)

(72) Inventors: Claudio Cropano, Zeiningen (CH); Dániel Árpád Carrera, Zeiningen (CH); Gavin Mager George, Zeiningen (CH); Leron Katsir, Zeiningen (CH); Maximilian Moritz Vogt, Zeiningen (CH); Mercedes Thieme, Zeiningen (CH); Michael Edward Ruckle, Zeiningen (CH); Michele Wyler, Zeiningen (CH)

(21) Appl. No.: 19/120,418

(22) PCT Filed: Oct. 13, 2023
(86) PCT No.: PCT/IB2023/060342

§ 371 Date: Apr. 11, 2025

(30) Foreign Application Priority Data
Oct. 13, 2022 (GB) 2215078.3

(51) Int. Cl. A01H 1/04; A01H 1/00; A01H 5/02; A01H 6/28; C12Q 1/6895

(52) U.S. Cl. CPC A01H 1/045; A01H 1/1215; A01H 5/02; A01H 6/28; C12Q 1/6895; C12Q 2600/13; C12Q 2600/156

(57) ABSTRACT

The invention relates to methods of identifying and characterizing a Cannabis spp. plant with respect to a flowering time trait comprising genotyping the plant for a quantitative trait locus (QTL) associated with a flowering time trait, and to methods of producing plants having a flowering time trait of interest based on defined allelic states of polymorphisms defining the QTL. Also provided are Cannabis spp. plants having a flowering time trait of interest comprising defined allelic states of polymorphisms defining the QTL and plants identified, characterized or produced by the methods described herein. The invention further relates to marker assisted selection and marker assisted breeding methods, in particular using a combination of specific markers provided, for obtaining plants having a flowering time trait of interest or for modulating the flowering time of cannabis plants.

### BACKGROUND OF THE INVENTION

The invention relates to methods of identifying and characterizing a Cannabis spp. plant comprising a quantitative trait locus (QTL) associated with flowering time, and to Cannabis spp. plants having a flowering time trait of interest comprising defined allelic states of the polymorphisms defining the QTL. The invention further relates to plants with a flowering time trait of interest identified by the methods described herein. The invention also relates to marker assisted selection and marker assisted breeding methods for obtaining plants having a flowering time trait of interest. Also provided are methods of producing Cannabis spp. plants with the flowering time trait of interest and plants produced by these methods, based on the allelic state of the QTLs.

Modern Cannabis is derived from the cross hybridization of three biotypes; Cannabis sativa L. ssp. indica, Cannabis sativa L. ssp. sativa, and Cannabis sativa L. ssp. ruderalis. Cannabis was divergently bred into two distinct, albeit tentative types, called Hemp and HRT (high-resin-type) Cannabis, respectively, which are typically used for different purposes. Hemp is primarily used for industrial purposes, for example in feed, food, seed, fiber, and oil production. Conversely, HRT cannabis is largely cultivated and bred for high concentrations of the pharmacological constituents, cannabinoids, derived from resin in the trichomes. Biomass, including the leaf and stem, of cannabis can also be an important source of cannabinoids.

Cannabis produces phytocannabinoids a class of terpenoid that can act as antagonists and agonists of mammalian endocannabinoid receptors. The pharmacological action is derived from this ability of phytocannabinoids to disrupt and mimic endocannabinoids. Due to its psychoactive properties, one cannabinoid, delta-9-tetrahydrocannabinol (THC), the decarboxylation product of the plant-produced delta-9-tetrahydrocannabinolic acid (THCA), has received much attention in illegal or unregulated breeding programs, with modern HRT varieties having THC concentrations of 0.5% to 30%.

The Cannabis Sativa L (cannabis) growing period comprises a vegetative phase followed by a reproductive flower development phase. These phases are sensitive to temperature and photoperiod. During long-day photoperiods, flowering is inhibited, and the plant will remain in a vegetative growth phase. Seasonal changes will induce flowering when day length reduces to a minimum of 10-12 hours of continuous darkness. This photoperiod-dependent growth pattern is typical among hemp-type and high-resin type cannabis. Certain varieties that can transition to the flowering phase independently of the photoperiod are referred to as “autoflowering” or “day-neutral”.

In cannabis, flowering time has been shown to be critical for flower quality, oil seed, and hemp fiber composition at harvest. Most cannabis varieties are short-day plants, in that flowering is initiated when the day length shortens to a critical photoperiod of approximately 10-12 hours. Cannabis varieties have been adapted through human selection and natural variation to a range of photoperiod and temperature regimes but only under inductive short-day conditions. Seasonal flowering of cannabis limits multiple harvests in outdoor cultivation and can lead to loss when plants flower late in the growing season where temperature compromises the harvest or when the transition to flowering is not initiated. High-resin-type cannabis that has been under selection pressure for cannabinoid production in indoor environments and has been neglected for selection based on flowering time. Indoor varieties under controlled lighting are selected for plants that mature early and are ready to be switched to the flowering phase artificially or for other criteria that give an advantage to the artificial cultivation system. This limits the success of indoor varieties when they are cultivated in outdoor conditions.

The human-driven introduction of crops into new climatic conditions, at different latitudes, altitudes, and temperatures, away from their natural ecosystems has required the selection of flowering time traits to maintain yield in these environments. Genetic studies to identify the mechanisms of photoperiod adaptation in a diverse range of crop species have found that the responsible genes are often involved in the regulation of the plant circadian clock. Understanding crop species' flowering pathways relies heavily on flowering time research conducted in Arabidopsis thaliana (Arabidopsis), a model long-day dicot plant.

In Arabidopsis, the perception of appropriate flowering conditions, the integration of a number of signals including temperature, photoperiod, developmental stage, and gibberellin signalling, leads to the expression of Flowering Locus T (FT), a member of the Phosphatidyl Ethanolamine Binding Protein (PEBP) family that are central to flower timing in plants.

In Arabidopsis, under long-day conditions, FT expression is, in part, driven by the accumulation of CONSTANS (CO)—a zinc finger-type transcription factor that is another key regulator of flowering. FT then translocates from the leaf to the apical meristem where it triggers the expression of floral meristem identity genes, including LEAFY and APETALA1, leading to flowering. Under short-day conditions, where CO levels are kept low, FT expression is restricted, and Arabidopsis flowering is delayed. An example of a regulator of flowering time in Arabidopsis is Agamous-like 24 (AGL24). Repression or overexpression of this gene in Arabidopsis results in late or early flowering, respectively.

In short-day plants, such as rice, induction of flowering occurs when the days shorten, as in cannabis. Heading date1 (Hd1), a CO homolog, responds to short-day photoperiod cues to activate Hd3a, a rice FT homolog. In long days, it has the opposite activity, acting as a repressor of Hd3a to suppress flowering.

Though the molecular basis of long-day and short-day plant flowering is not fully understood, photoperiod adaptions are economically important. Changes to heading date in cultivated rice varieties, for example that allow its cultivation in a range of climates, are largely driven by a variation to Hd1 and Hd3a. FT and CO, and their homologs in other plant species, play central roles in photoperiod regulation of flowering in long- and short-day plants, however the identities and roles of many more regulators that are integral to flowering time are yet to be identified.

In cannabis, little is known about the genes and mechanisms involved in flowering time regulation. Selection of cannabis plants for flowering time traits can be challenging for breeders, as plants must complete flowering to be evaluated for the phenotype. Breeding for flowering time can be complex because environmental factors can also influence flowering time. Traditional breeding strategies, while potentially useful, are slower and can result in the loss of favorable linked characteristics.

Because there are no good methods for selecting for flowering time cannabis seedlings based on morphological indicators early on, molecular markers would be useful for assessing flowering time at the seedling stage for incorporation into a breeding strategy or for selection of cannabis plants for production. As such, the identification of molecular markers to identify and select for and against cannabis plants that may have increased earlier, and later, flowering times would be a significant contribution to the cannabis industry. In addition to the above, control over flowering time could improve the potential geographic range of cannabis production.

In the present invention, several genetic regions in cannabis that significantly associate with cannabis flowering time were identified from a parent population displaying segregation of the flowering time trait and a method to identify the regions that contribute the most to the trait was employed, with the aim of developing varieties with a range of cannabis flowering times phenotype.

### SUMMARY OF THE INVENTION

The present invention describes methods of identifying and/or characterizing a Cannabis spp. plant with respect to a flowering time trait comprising genotyping the plant for at least one quantitative trait locus (QTL) associated with a flowering time trait, and to methods of producing plants having a flowering time trait of interest based on defined allelic states of polymorphisms defining the QTL. Also described are Cannabis spp. plants having a flowering time trait of interest comprising defined allelic states of polymorphisms defining the QTLs and plants identified, characterized or produced by the methods described. The invention further relates to marker assisted selection and marker assisted breeding methods, in particular using a combination of specific markers provided herein, for obtaining plants having a flowering time trait of interest or for modulating the flowering time of cannabis plants.

According to a first aspect of the present invention there is provided for a method for characterizing a Cannabis spp. plant with respect to a flowering time trait, the method comprising the steps of: (i) genotyping at least one plant with respect to at least one flowering time QTL by detecting one or more polymorphisms associated with the flowering time trait as defined in Table 2; and (ii) characterizing the one or more plants with respect to the at least one flowering time QTL as having an early flowering time QTL, a late flowering time QTL or an intermediate flowering time QTL based on the genotype at the polymorphism.

In a first embodiment of the method for characterizing a Cannabis spp. plant with respect to a flowering time trait, the polymorphism may be selected from the group consisting of “common_941”, “common_2630”, “common_3426”, “common_679”, and combinations thereof, as defined in Table 2. These markers have all been shown to have particularly high predictive value for the flowering time QTL and trait, particularly in combination.

In a second embodiment of the method for characterizing a Cannabis spp. plant with respect to a flowering time trait, the genotyping may be performed by any PCR-based detection method using molecular markers, by sequencing of PCR products containing the one or more polymorphisms, by targeted resequencing, by whole genome sequencing, or by restriction-based methods, for detecting the one or more polymorphisms.

According to a third embodiment of the method for characterizing a Cannabis spp. plant with respect to a flowering time trait, the molecular markers may be for detecting polymorphisms at regular intervals within the at least one flowering time QTL such that recombination can be excluded. In an alternative embodiment, the molecular markers may be for detecting polymorphisms at regular intervals within the at least one flowering time QTL such that recombination can be quantified to estimate linkage disequilibrium between a particular polymorphism and the flowering time phenotype. It will be appreciated by those of skill in the art that several possible markers may be designed for detecting the polymorphisms. For example, molecular markers may be for detecting polymorphisms such that recombination events can be detected to a resolution of 10,000 or 100,000 or 500,000 base pairs within the QTL. In one embodiment, the molecular markers may be designed based on a context sequence for the polymorphism as provided in Table 4 herein, or the molecular markers may be selected from the primer pairs as defined in Table 5.

In a fourth embodiment of the method for characterizing a Cannabis spp. plant with respect to a flowering time trait, the flowering time QTL may be selected from one or more QTLs defined in Table 3 with reference to the CS10 reference genome and defined by one or more polymorphisms associated with the flowering time trait as defined in Table 2, or a combination of such QTLs. In another embodiment, the flowering time QTL may be defined by a genetic marker linked to the QTL defined in Table 3.

According to a second aspect of the present invention, there is provided for a method of producing a Cannabis spp. plant having a flowering time trait of interest, the method comprising the steps of: (i) providing a donor parent plant having in its genome at least one flowering time QTL characterized by one or more polymorphisms associated with the flowering time trait of interest as defined Table 2; (ii) crossing the donor parent plant having the at least one flowering time QTL with at least one recipient parent plant to obtain a progeny population of cannabis plants; (iii) screening the progeny population of cannabis plants for the presence of the at least one flowering time QTL; and (iv) selecting one or more progeny plants having the at least one flowering time QTL, wherein the mature plant displays the flowering time trait of interest. The flowering time trait of interest may be an early flowering time trait, a late flowering time trait, or an intermediate flowering time trait. In this way, the trait can be modulated in a plant using the flowering time QTLs and markers therefor described herein.

In a first embodiment of the method of producing a Cannabis spp. plant having a flowering time trait of interest, the method may further comprise the steps of: (v) crossing the one or more progeny plants with the donor recipient plant; or (vi) selfing the one or more progeny plants.

According to a second embodiment of the method of producing a Cannabis spp. plant having a flowering time trait of interest, the screening may comprise genotyping at least one plant from the progeny population with respect to the at least one flowering time QTL by detecting one or more polymorphisms associated with the flowering time trait of interest as defined Table 2.

In a third embodiment of the method of producing a Cannabis spp. plant having a flowering time trait of interest, the method may comprise a step of genotyping the donor parent plant with respect to the at least one flowering time QTL by detecting one or more polymorphisms associated with the flowering time trait of interest as defined in Table 2, preferably prior to step (i).

According to a fourth embodiment of the method of producing a Cannabis spp. plant having a flowering time trait of interest, the genotyping may be performed by a PCR-based detection method using molecular markers, by sequencing of PCR products containing the one or more polymorphisms, by targeted resequencing, by whole genome sequencing, or by restriction-based methods, for detecting the one or more polymorphisms.

In a fifth embodiment of the method of producing a Cannabis spp. plant having a flowering time trait of interest, the molecular markers may be for detecting polymorphisms at regular intervals within the at least one flowering time QTL such that recombination can be excluded. In an alternative embodiment, the molecular markers may be for detecting polymorphisms at regular intervals within the at least one flowering time QTL such that recombination can be quantified to estimate linkage disequilibrium between a particular polymorphism and the flowering time trait of interest. For example, molecular markers may be for detecting polymorphisms such that recombination events can be detected to a resolution of 10,000 or 100,000 or 500,000 base pairs within the QTL. It will be appreciated by those of skill in the art that several possible markers may be designed for detecting the polymorphisms. In one embodiment, the molecular markers may be designed based on a context sequence for the polymorphism in Table 4 or may be selected from the primer pairs defined in Table 5.

According to a further embodiment of the method of producing a Cannabis spp. plant having a flowering time trait of interest, the at least one flowering time QTL is an early flowering time QTL, a late flowering time QTL, or an intermediate flowering time QTL defined by the allelic state of the polymorphisms as provided in Table 2. Of particular use in producing a Cannabis spp. plant having a flowering time trait of interest, are the polymorphisms selected from the group consisting of “common_941”, “common_2630”, “common_3426”, “common_679”, and combinations thereof, as defined in Table 2, which have been shown to have particularly high predictive value for the flowering time QTL and trait, and particularly to a combination of these polymorphisms.

According to a third aspect of the present invention there is provided for a method of producing a Cannabis spp. plant that has a flowering time trait of interest, the method comprising introducing at least one flowering time QTL characterized by one or more polymorphisms associated with the flowering time trait of interest as defined in Table 2 into a Cannabis spp. plant, wherein said flowering time QTL is associated with the flowering time trait of interest in the plant. In one embodiment, introducing the at least one flowering time QTL comprises crossing a donor parent plant having the at least one flowering time QTL characterized by one or more polymorphisms associated with the flowering time trait of interest with a recipient parent plant. In an alternative embodiment, introducing the at least one flowering time QTL characterized by one or more polymorphisms associated with the flowering time trait of interest comprises genetically modifying the Cannabis spp. plant. Several methods of genetic modification are known to those of skill in the art, including targeted mutagenesis, genome editing, and gene transfer. For example, a flowering time QTL comprising one or more of the polymorphisms associated with the flowering time trait of interest as defined in Table 2 herein may be introduced into a plant by mutagenesis and/or gene editing. In particular the methods of genetically modifying a plant may be selected from the group consisting of CRISPR-Cas9 targeted gene editing, heterologous gene expression using various expression cassettes; TILLING, and non-targeted chemical mutagenesis using e.g., EMS. For example, CRISPR-Cas9 targeted gene editing may be achieved using a guide RNA. Alternatively, a Cannabis spp. plant may be transformed with a cassette containing the flowering time QTL associated with the flowering time trait of interest or a part thereof, via any transformation method known in the art.

In a one embodiment of the method of producing a Cannabis spp. plant that has a flowering time trait of interest, the at least one flowering time QTL is selected from one or more QTLs defined in Table 3 with reference to the CS10 reference genome and defined by one or more polymorphisms associated with the flowering time trait as defined in Table 2, or a combination thereof. In another embodiment, the flowering time QTL may be defined by a genetic marker linked to the QTL defined in Table 3.

According to a fourth aspect of the present invention there is provided for a Cannabis spp. plant characterized according to the method for characterizing a Cannabis spp. plant with respect to a flowering time trat as described herein. In some embodiments, the Cannabis spp. plant characterized according to the method of a characterizing a Cannabis spp. plant having a flowering time trait of interest as described herein is not exclusively obtained by means of an essentially biological process.

In a fifth aspect of the present invention there is provided for a Cannabis spp. plant produced according to the method of a producing a Cannabis spp. plant having a flowering time trait of interest as described herein. In some embodiments, the Cannabis spp. plant produced according to the method of a producing a Cannabis spp. plant having a flowering time trait of interest as described herein is not exclusively obtained by means of an essentially biological process.

According to a further aspect of the present invention there is provided for a Cannabis spp. plant comprising at least one flowering time QTL characterized by one or more polymorphisms associated with the flowering time trait of interest as defined in Table 2. In some embodiments, the plant is not exclusively obtained by means of an essentially biological process.

According to another aspect of the present invention there is provided for a quantitative trait locus that controls a flowering time trait in Cannabis spp., wherein the quantitative trait locus is selected from one or more QTLs defined in Table 3 with reference to the CS10 reference genome and defined by one or more polymorphisms associated with the flowering time trait as defined in Table 2, or a genetic marker linked to the QTL. In some embodiments, the quantitative trait loci defined in Table 3 are provided as isolated nucleic acid molecule(s).

According to yet a further aspect of the present invention there is provided for a Cannabis spp. plant comprising one or more quantitative trait loci defined herein.

### BRIEF DESCRIPTION OF THE FIGURES

Non-limiting embodiments of the invention will now be described by way of example only and with reference to the following figures:

FIG. 1: Segregation of flowering time in the 24 F2 populations tested. The F2 population designation is shown on the X-axis each plot. The Y-axis is flowering time where 1 is the latest flowering and 7 is the earliest flowering based on scoring for flowering weekly.

FIG. 2: A Manhattan Plot representing the results of a GWA of flowering time of a combined Cannabis F2 population using the BLINK Model. Each box represents a separate chromosome, with the chromosome name above the plot and the position on the chromosome on the X-axis below, the Y-axis is the LOD score, −log 10(p).

FIG. 3: A Plot illustrating the selection of the model to fit the minimum number of markers used to predict the greatest variance in flowering time. The X-axis represents the number of marker used for prediction. The Y-axis displays the model fit measured using the Bayesian information criterion (BIC).

### SEQUENCES

The nucleic acid and amino acid sequences listed herein and in any accompanying sequence listing are shown using standard letter abbreviations for nucleotide bases, and the standard one or three letter abbreviations for amino acids. It will be understood by those of skill in the art that only one strand of each nucleic acid sequence is shown, but that the complementary strand is included by any reference to the displayed strand.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims, which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items. It is, however, contemplated as a specific embodiment of the present disclosure that the term “comprising” encompasses the possibility of no further members being present, i.e., for the purpose of such an embodiment “comprising” is to be understood as having the meaning of “consisting of”.

Methods are provided herein for identifying and obtaining plants having a flowering time trait of interest, using molecular marker detection. The inventors of the present invention have further produced and selected for cannabis plants having different flowering time traits of interest by crossing plants using marker assisted selection or breeding techniques to obtain cannabis plants with varying flowering times. Also demonstrated herein, the inventors were able to use genome wide association (GWA) to identify multiple QTLs linked to flowering time. The method provides evidence that the genetic basis of flowering time is polygenic.

A total of twenty (20) QTLs for flowering time were identified in the combined F2 populations tested.

Table 2 herein provides several single nucleotide polymorphisms (SNPs) which define the QTLs associated with a flowering time trait of interest. In some embodiments one or more of the identified SNPs can be used to incorporate the flowering time trait of interest from a donor plant, containing one or more of the QTLs associated with the trait of interest, into a recipient plant. For example, the incorporation of the flowering time trait of interest may be performed by crossing a donor parent plant to a recipient parent plant to produce plants containing a haploid genome from both parents. Recombination of these genomes provides F1 progeny where each haploid complement of chromosomes, of the diploid genome, is comprised of genetic material from both parents.

In some embodiments, methods of identifying one or more QTLs that are characterized by a haplotype comprising of a series of polymorphisms in linkage disequilibrium are provided. The QTLs each display limited frequency of recombination within the QTLs. Preferably the polymorphisms are selected from any one of Table 2 herein, representing the flowering time QTLs. Molecular markers may be designed for use in detecting the presence of the polymorphisms and thus the QTLs. Further, the identified QTL polymorphisms and the associated molecular markers may be used in a cannabis breeding program to predict the flowering time trait of interest of plants in a breeding population and can be used to produce cannabis plants that display the flowering time trait of interest, compared to a control population. The QTLs identified can be used to modulate flowering time in both high-resin-type (HRT) and hemp cannabis.

As used herein, reference to a plant or a variety with a “flowering time trait” refers to a plant or a variety that shows a variation in flowering time, conferred by the flowering time QTLs identified herein. In some cases, it may be desirable to obtain a plant displaying various flowering time traits of interest, where it is desired to obtain a plant displaying early flowering time, intermediate flowering time, or late flowering time trait. Different flowering time traits may impart a growth penalty on plant development, which can include, among others, an impact on plant growth, floral development, and yield of desirable cannabis products including flower, fiber, and seed. A “flowering time trait of interest” refers to the state of the plant with respect to the flowering time trait, and includes early flowering time, late flowering time, or an intermediate flowering time compared with a control population and as further described herein.

As used herein, reference to a plant or a variety's “flowering time” refers to the flowering of a plant or a variety or the appearance of pistils during the course of plant growth in a plant that undergoes seasonal flowering.

As used herein, reference to a plant or variety with an “early flowering time” refers to plant or variety that has a variation in seasonal flowering such that it flowers earlier in the season during the course of plant growth. Plants that display early flowering time on average flower earlier in the season compared to a control plant or a plant from a control population.

As used herein, reference to a plant or variety with a “late flowering time” refers to plant or variety that has a variation in seasonal flowering such that it flowers later in the season on average during the course of plant growth. Plants that display late flowering time on average flower later in the season compared to a control plant or a plant from a control population.

It is a particular aim of the present invention to identify and characterize a plant for the flowering time trait of interest early in the plant lifecycle, particularly prior to the plant displaying the flowering time trait of interest, or to produce a flowering time trait of interest into a breeding population of plants early on. This can be achieved by genotyping the plant using molecular markers for detecting at least one QTL associated with the flowering time trait of interest prior to the appearance of flowers.

As used herein a “quantitative trait locus” or “QTL” is a polymorphic genetic locus with at least two alleles that differentially affect the expression of a continuously varying phenotypic trait when present in a plant or organism which is characterised by a series of polymorphisms in linkage disequilibrium with each other.

As used herein, the term “flowering time QTL” or “flowering time quantitative trait locus” refers to a quantitative trait locus comprising part, or all, of the QTLs characterized by the polymorphisms having an allelic state associated with a flowering time trait of interest as described in Table 2, or characterized by combinations of said polymorphisms.

As used herein, the term “early flowering time QTL” or “early flowering time quantitative trait locus” refers to a quantitative trait locus characterized by one or more polymorphisms having an allelic state associated with an early flowering time as described or defined in Table 2.

As used herein, the term “late flowering time QTL” or “late flowering time quantitative trait locus” refers to a quantitative trait locus characterized by one or more polymorphisms having an allelic state associated with late flowering time described or defined in Table 2.

As used herein, the term “intermediate flowering time QTL” or “intermediate flowering time quantitative trait locus” refers to a quantitative trait locus characterized by one or more polymorphisms having an allelic state associated with intermediate flowering time described or defined in Table 2.

As used herein, “haplotypes” refer to patterns or clusters of alleles or single nucleotide polymorphisms that are in linkage disequilibrium and therefore inherited together from a single parent. The term “linkage disequilibrium” refers to a non-random segregation of genetic loci or markers. Markers or genetic loci that show linkage disequilibrium are considered linked.

As used herein, the term “flowering time haplotype” refers to the subset of the polymorphisms contained within any one of the early flowering QTLs which exist on a single haploid genome complement of the diploid genome, and which are in linkage disequilibrium with the flowering time trait.

As used herein, the term “donor parent plant” refers to a plant having a flowering time haplotype, or one or more flowering time alleles associated with the flowering time trait of interest.

As used herein, the term “recipient parent plant” refers to a plant having a flowering time haplotype, or one or more flowering time alleles, not associated with the flowering time trait of interest.

The term “flowering time allele” refers to the haplotype allele within a particular QTL that confers, or contributes to, the flowering time trait of interest, or alternatively, is an allele that allows the identification of plants with the flowering time trait of interest that can be included in a breeding program, particularly to select for the flowering time trait (“marker assisted breeding”, “marker assisted selection”, or “genomic selection”).

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same, or genetically identical plant). The term “crossing” refers to the act of fusing gametes via pollination to produce progeny.

The term “GWAS” or “Genome wide association study” or “GWA” or “Genome wide association” as used herein refers to an observational study of a genome-wide set of genetic variants or polymorphisms in different individual plants to determine if any variant or polymorphism is associated with a trait, specifically the flowering time trait, in particular the early flowering time trait.

As used herein a “polymorphism” is a particular type of variance that includes both natural and/or induced multiple or single nucleotide changes, short insertions, or deletions in a target nucleic acid sequence at a particular locus as compared to a related nucleic acid sequence. These variations include, but are not limited to, single nucleotide polymorphisms (SNPs), indel/s, genomic rearrangements, and gene duplications.

As used herein, the term “LOD score” or “logarithm (base 10) of odds” refers to a statistical estimate used in linkage analysis, wherein the score compares the likelihood of obtaining the test data if the two loci are indeed linked, to the likelihood of observing the same data purely by chance. The LOD score is a statistical estimate of whether two genetic loci are physically near enough to each other (or “linked”) on a particular chromosome that they are likely to be inherited together. A LOD score of 3 or higher is generally understood to mean that two genes are located close to each other on the chromosome. In terms of significance, a LOD score of 3 means the odds are 1,000:1 that the two genes are linked and therefore inherited together.

As used herein, the term “quantile-quantile” or “Q-Q” refers to a graphical method for comparing two probability distributions by plotting their quantiles against each other. If the two distributions being compared are similar, the points in the Q-Q plot will approximately lie on the line y=x. If the distributions are linearly related, the points in the Q-Q plot will approximately lie on a line, but not necessarily on the line y=x. Q-Q plots can also be used as a graphical means of estimating parameters in a location-scale family of distributions.

As used herein, a “causal gene” is the specific gene having a genetic variant (the “causal variant”) which is responsible for the association signal at a locus and has a direct biological effect on the flowering time trait. In the context of association studies, the genetic variants which are responsible for the association signal at a locus are referred to as the “causal variants”. Causal variants may comprise one or more “causal polymorphisms” that have a biological effect on the phenotype.

The term “nucleic acid” encompasses both ribonucleotides (RNA) and deoxyribonucleotides (DNA), including cDNA, genomic DNA, isolated DNA and synthetic DNA. The nucleic acid may be double-stranded or single-stranded. Where the nucleic acid is single-stranded, the nucleic acid may be the sense strand or the antisense strand. A “nucleic acid molecule” or “polynucleotide” refers to any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides. By “cDNA” is meant a complementary or copy DNA produced from an RNA template by the action of RNA-dependent DNA polymerase (reverse transcriptase).

In some embodiments, the nucleic acid molecules of the invention may be operably linked to other sequences. By “operably linked” is meant that the nucleic acid molecules, such as those comprising the QTLs of the invention, and regulatory sequences are connected in such a way as to permit expression of the proteins when the appropriate molecules are bound to the regulatory sequences. Such operably linked sequences may be contained in vectors or expression constructs which can be transformed or transfected into plant cells or plants for expression. A “regulatory sequence” refers to a nucleotide sequence located either upstream, downstream or within a coding sequence. Generally regulatory sequences influence the transcription, RNA processing or stability, or translation of an associated coding sequence. Regulatory sequences include but are not limited to: effector binding sites, enhancers, introns, polyadenylation recognition sequences, promoters, RNA processing sites, stem-loop structures, translation leader sequences and the like.

The term “promoter” refers to a DNA sequence that is capable of controlling the expression of a nucleic acid coding sequence or functional RNA. A promoter may be based entirely on a native gene, or it may be comprised of different elements from different promoters found in nature. Different promoters are capable of directing the expression of a gene at different stages of development, or in response to different environmental or physiological conditions. An “inducible promoter” is promoter that is active in response to a specific stimulus. Several such inducible promoters are known in the art, for example, chemical inducible promoters, developmental stage inducible promoters, tissue type specific inducible promoters, hormone inducible promoters, environment responsive inducible promoters.

The term “isolated”, as used herein means having been removed from its natural environment. Specifically, the nucleic acid(s) identified herein, for example a nucleic acid carrying one or more of the QTLs defined herein, may be isolated nucleic acids, which have been removed from plant material where they naturally occur.

The term “purified”, relates to the isolation of a molecule or compound in a form that is substantially free of contamination or contaminants. Contaminants are normally associated with the molecule or compound in a natural environment, purified thus means having an increase in purity as a result of being separated from the other components of an original composition. The term “purified nucleic acid” describes a nucleic acid sequence that has been separated from other compounds including, but not limited to polypeptides, lipids, and carbohydrates which it is ordinarily associated with in its natural state.

The term “complementary” refers to two nucleic acid molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” or “substantially homologous” sequence is a nucleotide sequence that differs from a reference sequence only by one or more conservative substitutions, or by one or more non-conservative substitutions, deletions, or insertions located at positions of the sequence that do not destroy or substantially alter the activity of the polypeptide encoded by the nucleic acid molecule. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” or “substantially homologous” if they hybridize under high stringency conditions. The “stringency” of a hybridization reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridization generally depends on the ability of denatured DNA to re-anneal when complementary strands are present in an environment below their melting temperature. A typical example of such “stringent” hybridization conditions would be hybridization carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

Nucleotide positions of polymorphisms described herein are provided with reference to the corresponding position on the Cannabis sativa (assembly cs10) representative genome, provided as RefSeq assembly accession: GCF_900626175.2 on NCBI, loaded on 14 Feb. 2019, referred to herein as “cs 10 reference genome” or “cs10 genome”.

### Methods of Identifying a QTL or Haplotype Responsible for Flowering Time, Particularly Early Flowering Time, and Molecular Markers Therefor

In some embodiments, methods are provided for identifying a QTL or haplotype responsible for a flowering time trait of interest, such as an early flowering time trait of interest, thereby to identify the QTL or haplotype responsible for the trait, and for selecting plants with the flowering time trait. In some embodiments, the methods may comprise the steps of:
a. Identifying a plant that displays an early flowering phenotype within a breeding program.
b. Establishing a population by crossing the identified plant to itself (selfing) or a recipient parent plant.
c. Genotyping the resultant F1, or subsequent populations, for example by sequencing methods.
d. Performing association studies, including phenotyping and linkage analysis, to discover QTLs and/or polymorphisms contained within the QTL.
e Optionally, identifying cannabis paralogs of previously characterized genes that may be involved in conferring the flowering time trait of interest.
f. Developing molecular markers that detect one or more polymorphisms linked to QTLs, alleles within these QTLs, or existing or induced polymorphisms.
g. Validating the molecular markers by determining the linkage disequilibrium between the marker and the early flowering trait.

### Trait Development and Introgression

In some embodiments, methods are provided for marker assisted breeding (MAB) or marker assisted selection (MAS) of plants having an flowering time QTL or displaying the flowering time trait of interest. The methods may comprise the steps of:
a. Identifying a plant that displays the flowering time trait of interest or phenotype or contains a flowering time QTL associated with the flowering time trait of interest as defined herein.
b. Establishing a population by crossing the identified plant to itself (selfing) or another recipient parent plant.
c. Genotyping and phenotyping the resultant F1, or subsequent, populations, for example by sequencing methods.
d. Performing association studies, inputting phenotype and genotype information to identify genomic regions enriched with polymorphisms associated with the flowering time trait of interest, to discover QTLs and/or polymorphisms contained within the QTL.
e. Optionally, identifying cannabis paralogs of previously characterized genes that may be involved in conferring the flowering time trait of interest.
f. Developing molecular markers that detect one or more polymorphisms linked to QTLs, alleles within these QTLs, or existing or induced polymorphisms.
g Using the molecular markers when introgressing the QTLs or polymorphisms into new or existing cannabis varieties to select plants containing the flowering time trait of interest or a flowering time haplotype associated with the flowering time trait of interest.

### QTLs and Marker Assisted Breeding

In some embodiments, during the breeding process, selection of plants displaying the flowering time trait of interest as described herein, may be based on molecular markers designed to detect polymorphisms linked to genomic regions that control the trait of interest by either an identified or an unidentified mechanism. Previously identified genetic mechanisms may, for example, have a direct or pleiotropic effect on the flowering time in a plant. In some embodiments, QTLs containing such elements are identified using association studies. Knowledge of the mode-of-action is not required for the functional use of these genomic regions in a breeding program. Identification of regions controlling unidentified mechanisms may be useful in obtaining plants with the flowering time trait of interest, based on identification of polymorphisms that are either linked to, or found within QTLs that are associated with the phenotype of the flowering time trait of interest using association studies.

### Construction of Breeding Populations

Breeding populations are the offspring of sexual reproduction events between two or more parents. The parent plants (F0) are crossed to create an F1 population each containing a chromosomal complement of each parent. In a subsequent cross (F2), recombination has occurred and allows for mostly independent segregation of traits in the offspring and importantly the reconstitution of recessive phenotypes that existed in only one of the parental lines.

According to some embodiments, QTLs that lead to the phenotype of the flowering time trait of interest are identified within synthetic populations of plants capable of revealing dominant, recessive, or complex traits. In one embodiment of the invention, a genetically diverse population of cannabis varieties, that are used to produce the synthetic population are integrate them into a breeding program by unnatural processes. In some embodiments, these processes result in changes in the genomes of the plants. The changes may include, but are not limited to, mutations and rearrangements in the genomic sequences, duplication of the entire genome (polyploidy), or activation of movement of transposable elements which may inactivate, activate or attenuate the activity of genes or genomic elements. According to one embodiment of the invention, the following methods are employed to integrate the plants into a breeding program include some or all of the following:
a. Growing plants in rich media or soils under artificial lighting;
b. Cloning of plants, often through a multitude of sub-cloning cycles;
c. Introduction of plants into in vitro, sterile growth environments, and subsequent removal to standard growth conditions;
d. Exposure to mutagens such as EMS, colchicine, silver nitrate, ethidium bromide, dinitroanilines, high concentrations mono or poly-chromatic light sources;
e. Growing plants under highly stressful conditions which include restricted space, drought, pathogen challenge, atypical temperatures, and nutrient stresses.

### Flowering Trait of Interest Association Studies and QTL Identification

In some embodiments, the synthetic populations created are either the offspring of the sexual reproduction or clones of plants in the breeding program such that genetic material of individuals in the synthetic populations is derived from one, or two, or more plants from the breeding program.

In one embodiment, plants identified within the synthetic population as having a flowering time trait of interest, such as the early flowering trait, may be used to create a structured population for the identification of the genetic locus responsible for the trait. The structured population may be created by crossing one (selfing) or more plants and recovering the seeds from those plants.

Plants in the structured population may be fully genotyped using genome sequencing to identify genetic markers for use in the association study (AS) database. Association mapping is a powerful technique used to detect quantitative trait loci (QTLs) specifically based on the statistical correlation between the phenotype and the genotype of the flowering time trait of interest. In a population generated by crossing, the amount of linkage disequilibrium (LD) is reduced between genetic marker and the QTL as a function of genetic distance in cannabis varieties with similar genome structures. Simple association mapping is performed by biparental crosses of two closely related lines where one line has a phenotype of interest, and the other does not. In some embodiments, advanced population structures may be used, including nested association mapping (NAM) populations or multi-parent advanced generation inter-cross (MAGIC) populations, however it will be appreciated that other population structures can also be effectively used. Biparental, NAM, or MAGIC structured populations can be generated and offspring, at F1 or later generations, may be maintained by clonal propagation for a desired length of time. In some embodiments, QTLs may be identified using the high-density genetic marker database created by genotyping the founder lines and structured population lines. This marker database may be coupled with an extensive phenotypic trait characterization dataset, including, for example, the flowering time phenotype of the plants. Using the association studies described herein, together with accurate phenotyping, this method is able to identify genomic regions, QTLs and even specific genes or polymorphisms responsible for the flowering time phenotype that are directly introduced into recipient lines. Polygenic phenotypes may also be identified using the methods described herein.

In one embodiment, the structured population is grown to the flowering stage. To characterize the phenotypes of the lines they are clonally reproduced so the phenotypic data can be collected in feasible replicates.

### Genomic Selection

In some embodiments, during the breeding process, selection of plants by genomic selection (GS) may be conducted. Genomic selection is a method in plant breeding where the genome wide genetic potential of an individual is determined to predict breeding values for those individuals. In some embodiments, the accuracy of genomic selection is affected by the data used in a GS model including size of the training population, relationships between individuals, marker density, use of pedigree information, and inclusion of known QTLs.

In some embodiments, a QTL or a SNP known to be associated with a trait that contributes to selection criteria can improve the accuracy of genomic selection models. In some embodiments, a genomic selection model that incorporates flowering time can be improved by the inclusion of the flowering time QTLs in the GS model. In some embodiments, the SNPs described in Table 2 may be useful in a genomic selection model, for example where genotypes with unknown phenotypes are evaluated using an approach like a random forest algorithm for prediction of the pathogen resistance trait, and particularly in combination, to improve the predictive power of the model.

### Molecular Markers to Detect Polymorphisms

As used herein, the term “marker” or “genetic marker” refers to any sequence comprising a particular polymorphism or haplotype described herein that is capable of detection. For example, a marker may be a binding site for a primer or set of primers that is designed for use in a PCR-based method to amplify and thus detect a polymorphism or haplotype. Alternatively, the marker may introduce a restriction enzyme recognition site, or result in the removal of a restriction enzyme recognition site. Plants can be screened for a particular trait based on the detection of one or more markers confirming the presence of the polymorphism. Marker detection systems that may be used in accordance with the present invention include, but are not limited to polymerase chain reaction (PCR) followed by sequencing, Kompetitive allele specific PCR (KASP), restriction fragment length polymorphisms (RFLPs) analysis, amplified fragment length polymorphisms (AFLPs), cleaved amplified polymorphic sequences (CAPS), or any other markers known in the art.

In some embodiments “molecular markers” refers to any marker detection system, including primers designed based on the context sequences for the polymorphisms identified herein, provided in Table 4. Such molecular markers may include PCR primers, or targeted sequencing primers such as those described in the examples below, more specifically the primers defined in Table 5.

For example, PCR primers may be designed that consist of a reverse primer and two forward primers that are homologous to the part of the genome that contains a polymorphism but differ in the 3′ nucleotide such that the one primer will preferentially bind to sequences containing the polymorphism and the other will bind to sequences lacking it. The three primers are used in single PCR reactions where each reaction contains DNA from a cannabis plant as a template. Fluorophores linked to the forward primers provide, after thermocycling, a different relative fluorescent signal for homozygous and heterozygous alleles containing the polymorphism and for those lacking the polymorphism, respectively.

In some embodiments, allele-specific primers may each harbour a unique tail sequence that corresponds with a universal FRET (fluorescence resonant energy transfer) cassette. For example, the primer specific to the SNP may be labelled with a FAM and the other specific primer with a HEX dye. During the PCR thermal cycling performed with these primers, the allele-specific primer binds to the genomic DNA template and elongates, so attaching the tail sequence to the newly synthesized strand. The complement of the allele-specific tail sequence is then generated during subsequent rounds of PCR, enabling the FRET cassette to bind to the DNA. Alleles are discriminated through the competitive binding of the two allele-specific forward primers. At the end of the PCR reaction a fluorescent plate is read using standard tools which may include RT-PCR devices with the capacity to detect florescent signals and is evaluated with commercial software.

If the genotype at a given polymorphism site is homozygous, one of the two possible fluorescent signals will be generated. If the genotype is heterozygous, a mixed fluorescent signal will be generated. By way of example, genomic DNA extracted from cannabis leaf tissue at seedling stage can be used as a template for PCR amplifications with reaction mixtures containing the three primers. Final fluorescent signals can be detected by a thermocycler and analyzed using standard software for this purpose, which discriminates between individuals that are heterozygotes or homozygotes for the polymorphism.

In some embodiments, molecular markers to one, two or more of the SNPs in the haplotype can be used to identify the presence of the QTL and by association, the flowering time trait of interest.

Further, the QTL may include a number of individual polymorphisms in linkage disequilibrium, which constitute a haplotype and which, with high frequency, can be inherited from a donor parent plant as a unit. Therefore, in some embodiments, molecular markers can be utilized which have been designed to identify numerous polymorphisms which are in linkage disequilibrium with other polymorphisms, any of which can be used to effectively predict the phenotype of the offspring for the flowering time trait of interest.

According to some embodiments, any polymorphism in linkage disequilibrium with one or more of the flowering time QTLs can be used to determine the flowering time haplotype in a breeding population of plants, as long as the polymorphism is unique to the flowering time trait of interest in the donor parent plant when compared to the recipient parent plant.

In some embodiments, the desired trait may be the early flowering time trait, and the donor parent plant may be a plant that has been genetically modified or selected to include an early flowering time QTL defined by a polymorphism or allele conferring the early flowering time trait, for example any, some, or all of the polymorphisms or alleles defined in Table 2 associated with the trait.

Alternatively, the desired trait may be the intermediate- or late flowering time trait, and the donor parent plant may be a plant that has been genetically modified or selected to include a an intermediate- or late flowering time QTL defined by a polymorphism or allele associated with intermediate- or late flowering time, for example any, some, or all of the polymorphisms or alleles defined in Table 2 associated with the trait.

In some embodiments, donor parent plants, as described above, are used as one of two parents to create breeding populations (F1) through sexual reproduction. In this embodiment, donor parent plants may be identified by detecting polymorphisms using the molecular markers as described above.

Methods for reproduction that are known in the art may be used. The donor parent plant provides the flowering time trait of interest to the breeding population. The trait is made to segregate through the population (F2) through at least one additional crossing event of the offspring of the initial cross. This additional crossing event can be either a selfing of one of the offspring or a cross between two individuals, provided that each plant used in the F1 cross contains at least one copy of a desired QTL allele or haplotype.

In some embodiments, the flowering time allele or flowering time haplotype, such as the early flowering time allele or the haplotype associated with the early flowering time trait in plants to be used in the F1 cross is determined using the described molecular markers. In some embodiments, the resulting F2 progeny, or subsequent progeny, is/are screened for any of the flowering time polymorphisms, and in particular early flowering time polymorphisms, described herein.

The plants at any generation can be produced by asexual means like cutting and cloning, or any method that yields a genetically identical offspring.

Production of Cannabis Spp. Plants Having the Early Flowering Time Trait

In some embodiments, a Cannabis spp. plant that has the late flowering time trait or an intermediate flowering time trait may be converted into a plant having an early flowering time trait according to the methods of the present invention by providing a breeding population where the donor parent plant contains an early flowering time QTL associated with an early flowering time trait and the recipient parent plant either displays the late- or intermediate flowering time phenotype or contains the late- or intermediate flowering time QTL.

In some embodiments the late- or intermediate flowering time phenotype may be removed from a recipient parent plant by crossing it with a donor parent plant having the early flowering time QTL. In some embodiments the donor parent plant has an early flowering time phenotype and a contains a contiguous genomic sequence characterized by one or more of the polymorphisms of Table 2 associated with the early flowering time allele or haplotype.

In some embodiments, the donor parent plant is any cannabis variety that is cross fertile with the recipient parent plant.

In some embodiments, MAS or MAB may be used in a method of backcrossing plants carrying the early flowering time trait to a recipient parent plant. For example, an F1 plant from a breeding population can be crossed again to the recipient parent plant. In some embodiments, this method is repeated.

In some embodiments, the resulting plant population is then screened for the early flowering time trait using MAS with molecular markers to identify progeny plants that contain one or more polymorphism, such as any of those described Table 2, indicating the presence of an allele of a QTL associated with the early flowering time phenotype. In another embodiment, the population of cannabis plants may be screened by any analytical methods known in the art to identify plants with desired characteristics, specifically the early flowering time trait.

Production of Cannabis Spp. Plants Having a Late- or Intermediate Flowering Time Trait

In some embodiments, a Cannabis spp. plant that has the early flowering time trait may be converted into a plant having an intermediate- or late flowering time trait according to the methods of the present invention by providing a breeding population where the donor parent plant contains a late- or intermediate flowering time QTL and the recipient parent plant either displays the early flowering time phenotype or contains the early flowering time QTL.

In some embodiments the early flowering time phenotype may be removed from a recipient parent plant by crossing it with a donor parent plant having the late- or intermediate flowering time QTL. In some embodiments the donor parent plant has a late- or intermediate flowering time phenotype and contains a contiguous genomic sequence characterized by one or more of the polymorphisms of Table 2 associated with the late flowering time allele or haplotype, or the intermediate flowering time allele or haplotype.

In some embodiments, the donor parent plant is any cannabis variety that is cross fertile with the recipient parent plant.

In some embodiments, MAS or MAB may be used in a method of backcrossing plants carrying the late- or intermediate flowering time trait to a recipient parent plant. For example, an F1 plant from a breeding population can be crossed again to the recipient parent plant. In some embodiments, this method is repeated.

In some embodiments, the resulting plant population is then screened for the late- or intermediate flowering time trait using MAS with molecular markers to identify progeny plants that contain one or more polymorphism, such as any of those described Table 2, indicating the presence of an allele of a QTL associated with the late- or intermediate flowering time phenotype. In another embodiment, the population of cannabis plants may be screened by any analytical methods known in the art to identify plants with desired characteristics, specifically the late- or intermediate flowering time trait.

### Methods to Genetically Engineer Plants to Achieve the Flowering Time Trait of Interest Using Mutagenesis or Gene Editing Techniques

Identifying QTLs, and individual polymorphisms, that correlate with a trait when measured in an F1, F2, or similar, breeding population indicates the presence of one or more causative polymorphisms in close proximity the polymorphism detected by the molecular marker. In some embodiments, the polymorphisms associated with the flowering time trait of interest are introduced into a plant by other means so that the trait can be introduced into plants that would not otherwise contain associated causative polymorphisms or removed from plants that would otherwise contain associated causative polymorphisms. For example, the polymorphisms detailed in Table 2 are molecular markers that can be used to indicate the presence of a possible causative polymorphism.

The entire QTLs or parts thereof which confer the flowering time trait of interest described herein may be introduced into the genome of a cannabis plant to obtain plants with early flowering time, late flowering time or intermediate flowering time, through a process of genetic modification known in the art, for example, but not limited to, heterologous gene expression using an expression cassette including a sequence encoding the QTL(s) or part thereof, or the nucleic acids comprising them. The expression cassette may contain all or part of the QTL(s), including possible causative polymorphisms.

The flowering time trait of interest described herein may be introduced into the genome of a Cannabis spp. plant to obtain plants that exclude or include the causative polymorphisms and the potential to display a desired flowering time trait of interest through processes of genetic modification known in the art, for example, but not limited to, CRISPR-Cas9 targeted gene editing, TILLING, non-targeted chemical mutagenesis using e.g., EMS.

The present invention further provides methods for producing a modified Cannabis plant using genome editing or modification techniques. For example, genome editing can be achieved using sequence-specific nucleases (SSNs) the use of which results in chromosomal changes, such as nucleotide deletions, insertions or substitutions at specific genetic loci, particularly those associated with the flowering time trait of interest as described in Table 2 or a causative polymorphism linked thereto. Non limiting examples of SSNs include zinc finger nucleases (ZFNs), TAL effector nucleases (TALENs), meganuclease, and, clustered regularly interspaced short palindromic repeats (CRISPR)/CRISPR-associated protein (Cas) system. In some embodiments, non-limiting examples of Cas proteins suitable for use in the methods of the present invention include CsnI, CpfI Cas9, Cas 12, Cas 13, Cas 14, CasX and combinations thereof. In one embodiment, a modified Cannabis spp. plant with a flowering time trait of interest is generated using CRISPR/Cas9 technology, which is based on the Cas9 DNA nuclease guided to a specific DNA target by a single guide RNA (sgRNA). For example, the genome modification may be introduced using guide RNA, e.g., single guide RNA (sgRNA) designed and targeted to introduce a polymorphism associated with the flowering time trait of interest, such as a polymorphism associated with the flowering time trait of interest described in Table 2, or a causative polymorphism linked thereto.

DNA introduction into the plant cells can be performed using Agrobacterium infiltration, virus-based plasmid delivery of the genome editing molecules, and mechanical insertion of DNA (PEG mediated DNA transformation, biolistics, etc.). In some embodiments, the Cas9 protein may be directly inserted together with a gRNA (ribonucleoprotein-RNP's) in order to bypass the need for in vivo transcription and translation of the Cas9+gRNA plasmid in planta to achieve gene editing. In one embodiment, a genome edited plant may be developed and used as a rootstock, so that the Cas protein and gRNA can be transported via the vasculature system to the top of the plant and create the genome editing event in the scion.

According to one embodiment of the present invention, the method of genetically modifying a plant may be achieved by combining the Cas nuclease (e.g., Cas9, Cpf 1) with a predefined guide RNA molecule (gRNA). The gRNA is complementary to a specific DNA sequence targeted for editing in the plant genome and which guides the Cas nuclease to a specific nucleotide sequence. The gRNA may be designed based on the context sequence for the polymorphisms provided in Table 4. The predefined gene specific gRNA's may be cloned into the same plasmid as the Cas gene and this plasmid is inserted into plant cells as described above.

In some embodiments, once the guide RNA molecule and Cas9 nuclease reach the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which introduce a mutation at the cleavage site.

In one embodiment, a deletion form of the mutation may consist of at least 1 base pair deletion. As a result of this base pair deletion the gene coding sequence for a gene responsible for the flowering time trait of interest is disrupted and the translation of the encoded protein is compromised either by a premature stop codon or disruption of a functional or structural property of the protein.

In another embodiment, a flowering time trait of interest in Cannabis spp. plants may be introduced by generating gRNA with homology to a specific site of predetermined genes in the Cannabis genome or the QTLs defined herein. This gRNA may be sub-cloned into a plasmid containing the Cas9 gene, and the plasmid inserted into the Cannabis plant cells. In this way site specific mutations in the QTLs are generated, particularly causative polymorphisms linked to a SNP associated with the flowering time trait of interest described in Table 2, thus effectively modulating, including shortening or lengthening, the flowering time in the genome edited plant.

In some embodiments, a modified Cannabis spp. plant exhibiting early flowering time may be obtained using the targeted genome modification methods described above, wherein the plant comprises a targeted genome modification to introduce one or more polymorphisms associated with the early flowering time trait, particularly a causative polymorphism linked to a SNP defined in Table 2, wherein the modification effects an early flowering time trait.

In some embodiments, the genetic modification may be introduced using gene silencing, a process by which the expression of a specific gene product is lessened or attenuated. Gene silencing can take place by a variety of pathways, including by RNA interference (RNAi), an RNA dependent gene silencing process. In one embodiment, RNAi may be achieved by the introduction of small RNA molecules, including small interfering RNA (siRNA), microRNA (miRNA) or short hairpin RNA (shRNA), which act in concert with host proteins (e.g., the RNA induced silencing complex, RISC) to degrade messenger RNA (mRNA) in a sequence-dependent fashion. Such RNAi molecules may be designed based on the sequence of these genes. These molecules can vary in length (generally 18-30 base pairs) and may contain varying degrees of complementarity to their target mRNA in the antisense strand. Some, but not all, RNAi molecules have unpaired overhanging bases on the 5′ or 3′ end of the sense strand and/or the antisense strand. As used herein, the term “RNAi molecule” includes duplexes of two separate strands, as well as single strands that can form hairpin structures comprising a duplex region. The RNAi molecules may be encoded by DNA contained in an expression cassette and incorporated into a vector. The vector may be introduced into a plant cell using Agrobacterium infiltration, virus-based plasmid delivery of the vector containing the expression cassette and/or mechanical insertion of the vector (PEG mediated DNA transformation, biolistics, etc.).

Plants may be screened with molecular markers as described herein to identify transgenic individuals with early-, intermediate-, or late flowering time QTL or polymorphism(s), following the genetic modification.

In some embodiments, Cannabis spp. plants having one or more of the polymorphisms of Table 2 associated with early-, intermediate-, or late flowering time QTLs or a causative polymorphism linked thereto are provided. The polymorphisms may be introduced, for example, by genetic engineering. In some embodiments the one or more polymorphisms associated with the flowering time of interest or linked thereto are introduced into the plants by breeding, such as by MAS or MAB, for example as described herein.

The nucleic acid molecules comprising the early-, intermediate-, or late flowering time QTLs defined herein responsible for conferring an early-, intermediate-, or late flowering time trait, may be under the control of, or operably linked to, a promoter, for example an inducible promoter. Such nucleic acid molecules may be operably linked to the inducible promoter so as to induce or suppress the flowering time trait or phenotype in the plant or plant cell.

Accordingly, in a further embodiment, Cannabis spp. plants comprising an early-, intermediate-, or late flowering time QTL described herein, or one or more polymorphisms associated therewith, are provided. In some cases, such plants are provided for with the proviso that the plant is not exclusively obtained by means of an essentially biological process.

The following examples are offered by way of illustration and not by way of limitation.

### Example 1

### Genome-Wide Association Studies (GWAS) of Flowering Time in Cannabis

To identify molecular markers that contribute to early and late flowering in cannabis a diverse population of cannabis was collected and grown in a field trial in 2020 in Niederwil, Switzerland. Genotypes that displayed diverse flowering times, including early and late flowering times were used to generate F2 populations. Two populations, GID 21002057 and GID 21002025, that are high THC varieties were included in this study because the inventors reasoned that they had not undergone selection for early flowering due to their likely cultivation and selection for indoor environments. The inventors reasoned that by crossing high THC varieties with hemp varieties to generate segregating F2 populations they could identify novel flowering time traits. During outdoor field trials in 2021 these F2 populations, predicted to show segregation of the flowering time trait, were grown and monitored for flowering time.

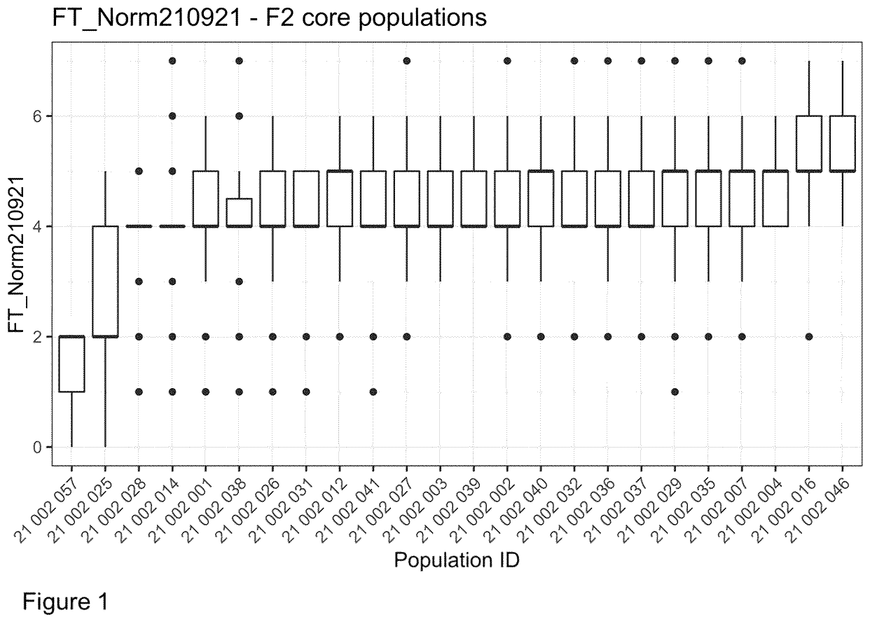

The inventors sought to understand the genetic basis for flowering time by recording the flowering time of 24 designed F2 populations, comprising in total 2338 individuals (Table 1). During the 2021 field trial season, flowering was first recorded in the field on Aug. 3, 2021. From then flowering time was scored once per week for seven weeks, until Sep. 21, 2021, when all plants in the F2 populations had initiated flowering. Flowering was noted after the presence of pistils were detected. If a plant was found to be flowering, it was scored with a 1, if not yet flowering, it was scored with a 0. This was repeated until all plants had flowered on Sep. 21, 2021. Plants that had not flowered by this date were not included in the study. The sum of the scores was calculated for each plant where a score of 1 indicated the latest flowering and a 7 indicates a plant that was earliest to flower. Looking at the individual populations, a range of flowering times can be observed (FIG. 1). Notably the populations characterized show on average a range of flowering time that can be described as early, intermediate and late flowering. Most populations observed were majorly intermediate flowering, between 3-4 weeks later than the first flowering event observed in any population, in the range described. However, populations GID 21002046 and 21002016 were comprised of plants where the distribution on average can be described as early flowering, where on average these plants developed flowers within the first two weeks after the first flowering event. The latest flowering populations GID 21002057 and GID 21002025 displayed an average flowering time that was 4-7 weeks later than the first flowering event. Interestingly, populations GID 21002057 and GID 21002025, as predicted, showed later flowering representing a validation of the approach to identify novel QTLs for flowering time by inclusion of high THC varieties that are typically not cultivated with respect to seasonal variation and add greater variation to the ranges of flowering times. An overview of the distribution of flowering time in the 24 F2 populations is shown in FIG. 1 and Table 1.

TABLE 1
An overview of the F2 populations used including the average
(“Mean”) flowering time (a score from 1-7, where 1
represents the latest flowering and 7 represents the earliest
flowering during the recorded period), the standard deviation
of the average flowering time (“StDev”), and the population
size given by the number of plants (“Number”).

| F2 Populations | Number | Mean flowering time | Standard deviation |
|---|---|---|---|
| 21 002 001 0000 | 163 | 3.98 | 1.13 |
| 21 002 002 0000 | 124 | 4.48 | 0.85 |
| 21 002 003 0000 | 118 | 4.38 | 0.58 |
| 21 002 004 0000 | 112 | 4.82 | 0.6 |
| 21 002 007 0000 | 73 | 3.71 | 0.99 |
| 21 002 012 0000 | 141 | 4.32 | 0.95 |
| 21 002 014 0000 | 81 | 3.95 | 1.23 |
| 21 002 016 0000 | 86 | 5.02 | 0.89 |
| 21 002 025 0000 | 86 | 2.35 | 1.41 |
| 21 002 026 0000 | 96 | 4.14 | 0.92 |
| 21 002 027 0000 | 95 | 4.34 | 1.1 |
| 21 002 028 0000 | 112 | 3.85 | 1 |
| 21 002 029 0000 | 101 | 4.64 | 1.1 |
| 21 002 031 0000 | 80 | 4.28 | 0.94 |
| 21 002 032 0000 | 91 | 4.51 | 0.82 |
| 21 002 035 0000 | 151 | 4.68 | 0.83 |
| 21 002 036 0000 | 116 | 4.55 | 0.82 |
| 21 002 037 0000 | 86 | 4.62 | 0.83 |
| 21 002 038 0000 | 111 | 4.05 | 0.98 |
| 21 002 039 0000 | 110 | 4.42 | 0.51 |
| 21 002 040 0000 | 101 | 4.49 | 1.05 |
| 21 002 041 0000 | 94 | 4.33 | 1.13 |
| 21 002 046 0000 | 118 | 5.26 | 0.97 |
| 21 002 057 0000 | 94 | 1.63 | 0.59 |

Following scoring for flowering time, All 2338 F2 plants described in Table 1 were sequenced. DNA was extracted from about 70 mg of leaf discs from all the plants evaluated using an adapted kit with “sbeadex” magnetic beads by LGC Genomics, which was automated on a KingFisher Flex with 96 Deep-Well Head by Thermo Fisher Scientific. The extracted DNA served as a template for the subsequent library preparation for sequencing. The library pools were prepared according to the manufacturer's instructions (AgriSeq™ HTS Library Kit-96 sample procedure from Thermo Fisher Scientific). Targeted sequencing of a custom SNP marker panel based on the Cannabis Sativa CS10 reference genome was carried out on the Ion Torrent system by Thermo Fisher Scientific. The primers for the SNPs identified are provided in Table 5 below. The library pool was loaded onto Ion 550 chips with Ion Chef and sequenced with Ion GeneStudio S5 Plus according to the manufacturer's instructions (Ion 550™ Kit from Thermo Fisher Scientific).

Using the 24 combined F2 populations with a total of 2338 individuals, a genome-wide association study (GWAS) was performed to detect significant associations between genotypic information derived from targeted resequencing of the custom SNP marker panel described above with flowering time. The flowering time was scored from 1-7, where 1 indicates on average late flowering and 7 indicates on average early flowering, were used as an input for GWAS.

The genotypic matrix was filtered for SNPs having more than 30% missing values within the population and a minor allele frequency lower than 5%. This resulted in 3627 SNP markers after filtering.

For better modelling the inventors reasoned that a high rate of missing values may be impacting the estimation of population structure and kinship among individuals. To solve this, an additional step was incorporated. The GWA with all F2 populations combined was performed again but instead used a SNP matrix that underwent a round of imputation for reducing the number of missing values. In order to reduce missing data in the genotype file, an imputation has been performed using the HapMap_imputation software (GitHub-mwylerCH/HapMap_Imputation). Briefly, the genotype file is converted to a hapmap format (comma separated, http://augustogarcia.me/statgen-esaIg/Hapmap-and-VCF-formats-and-its-integration-with-onemap/#hapmap).

In a first step, HapMap_Imputation counts the occurrence of each nucleotide at every single genotyped position. The most common nucleotide is defined as major allele, the second is defined as minor allele. Missing genotyping information is excluded. In the case major and minor alleles occur at the same number, the nucleotide of the reference cs10 genome (available as GCF_900626175.1 on NCBI) is chosen as major allele. Subsequently, HapMap_Imputation sorts markers by position and parses the hapmap into the required fastPHASE (Scheet & Stephens, 2006) input format. Briefly, HapMap_Imputation splits the haplotypes into two separate rows, converts major and minor alleles into 0 and 1 respectively and produces temporary files for each chromosome.

During the third step, HapMap_Imputation downloads the latest fastPHASE version and runs the imputation using 8 cores in parallel, fastPHASE is run with ten random starts of the imputation algorithm. After imputation, HapMap_Imputation reverses the 0 and 1 coding into the major and minor nucleotide, respectively. Subsequently, the two haplotypes are combined, and the separate chromosomes are merged into a single file.

The imputed genotypic matrix was filtered for SNPs having more than 30% missing values within the population and a minor allele frequency lower than 5%. This resulted in 5077 SNP markers after filtering, a significant increase in the number of SNP markers for association compared to non-imputed. The GWAS was performed using GAPIT version 3 (J. Wang & Zhang, 2021) with five statistical models: General Linear Model (GLM), Mixed Linear Model (MLM), FarmCPU and Blink. A quantile-quantile plot (QQ plot) was used to evaluate the statistical models. The Blink model performed the best by the inventors' evaluation and was used for the analysis. SNPs surpassing a LOD (−log₁₀(p-value)) value of 5 were considered to have a significant association with trait variation.

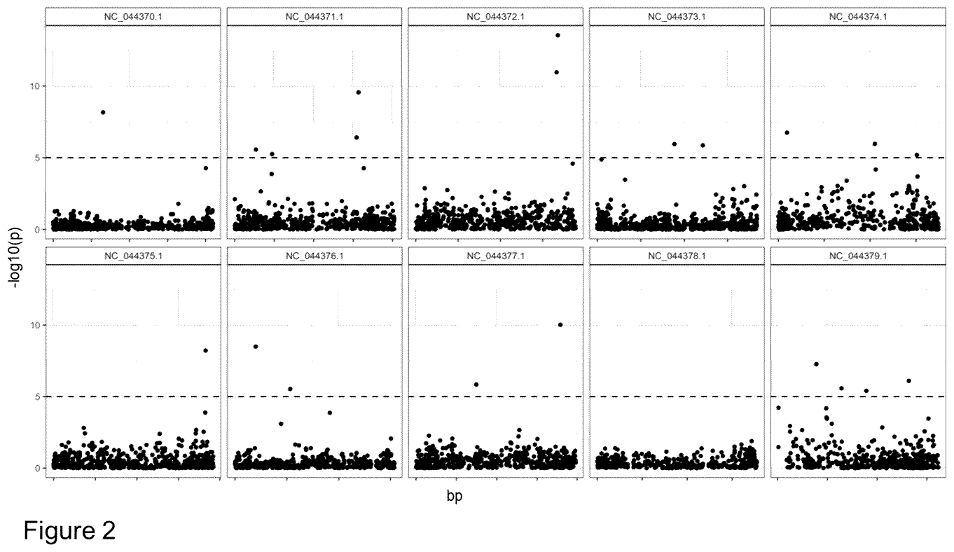

SNPs showing a significant association with flowering time, with an LOD value greater than 5 in the BLINK model in the imputed GWA, were found on most chromosomes with reference to the Cannabis Sativa CS10 genome, represented in a Manhattan plot shown in FIG. 2. The GWA identified significant SNPs associated with flowering time comprising 20 QTLS, QTL1-20 (Table 2 and Table 3).

The allelic variation of each SNP is associated with an average flowering time representative of the flowering times where that allelic variant is found in the plants that comprise the 24 F2 populations in Table 2. The allelic variant for each SNP listed in Table 2 is associated with early and late flowering time are listed along with their position and reference sequence. Interestingly, for each significant SNP identified as associated with flowering time, the heterozygous allele shows an intermediate flowering time phenotype as compared to the homozygous alleles, indicating this may a semi-dominant trait.

As an example, the inventors identified a locus associated with flowering time on chromosome NC_044371.1 QTL1 at position 32633325 defined here by the identified SNPs in Table 2. A large allelic variation in flowering time for an associated SNP can be useful in identifying QTLs that are major drivers of flowering time variation in Cannabis. The SNP that shows the greatest allelic variance in average flowering time, 1.83 weeks, is “common_941” of QTL4. In QTL 4, when “common_941” is Allele 1 (AA)—the flowering time is predicted to be on average 1.83 weeks earlier as compared to when “common_941” is Allele 3 (GG). The allelic variation for each associated SNP is given in Table 2.

The reference or context sequence for each of the SNPs identified is provided in Table 4 with reference to the CS10 genome. In Table 5, PCR primers designed to amplify each of the regions containing these SNPs, with reference to the CS10 genome, are provided in order for the allelic variant to be determined.

TABLE 2
SNPs associated with variation in flowering time (FT) in the F2 populations. The positions
and chromosome of the SNPs are provided with reference to the CS10 reference genome as
described herein. The LOD score is provided for the BLINK model as LOD. “Mean 1”, “Mean
2” and “Mean 3” denotes the average phenotypic value associated with Allele
1, Allele 2, and Allele 3, respectively based on scoring for flowering time from 1 to
7, where 1 represents the latest flowering and 7 represents the earliest flowering during
the recorded period, respectively. The average variance of the means between the allelic
variants for each SNP is given as variance (Var). The SNP position on the chromosome (Chr)
is provided with reference to the CS10 reference genome. “Count 1”, “Count 2”
and “Count 3” refers to the number of plants having Allele 1, Allele 2 and Allele 3, respectively.

| SNP | Chromosome | Position | LOD | Allele 1 | Allele 2 | Allele 3 | Mean 1 |
|---|---|---|---|---|---|---|---|
| common_1631 | NC_044372.1 | 83988588 | 13.5436245 | AA | AG | GG | 4.08235294 |
| common_1626 | NC_044372.1 | 83254930 | 10.9602244 | AA | AG | GG | 4.10989011 |
| common_4470 | NC_044377.1 | 71514027 | 10.0239918 | AA | AT | TT | 4.12871287 |
| common_946 | NC_044371.1 | 78310503 | 9.56453964 | AA | AG | GG | 3.99118644 |
| common_3557 | NC_044376.1 | 7991454 | 8.50260229 | AA | AC | CC | 4.14305365 |
| common_3426 | NC_044375.1 | 91437428 | 8.22096794 | AA | AC | CC | 4.47772277 |
| common_299 | NC_044370.1 | 32633325 | 8.16925535 | AA | AG | GG | 4.21069349 |
| common_5100 | NC_044379.1 | 15575449 | 7.27068923 | AA | AG | GG | 4.18188567 |
| common_2434 | NC_044374.1 | 4747237 | 6.75412097 | AA | AG | GG | 4.23748863 |
| common_941 | NC_044371.1 | 77104557 | 6.41415175 | AA | AG | GG | 4.67493797 |
| common_5391 | NC_044379.1 | 52728955 | 6.10473598 | AA | AC | CC | 4.04694168 |
| common_2630 | NC_044374.1 | 52581732 | 5.97191993 | AA | AG | GG | 3.59574468 |
| common_2031 | NC_044373.1 | 44346866 | 5.95661196 | AA | AG | GG | 4.32837055 |
| GBScompat_{—} | NC_044373.1 | 60699429 | 5.86969061 | AA | AG | GG | 4.26830467 |
| common_412 | | | | | | | |
| common_4191 | NC_044377.1 | 29886295 | 5.85007171 | AA | AC | CC | 4.25395257 |
| common_5139 | NC_044379.1 | 25700349 | 5.58472518 | AA | AC | CC | 4.06445993 |
| common_679 | NC_044371.1 | 13434356 | 5.57459516 | AA | AG | GG | 4.33301344 |
| GBScompat_{—} | NC_044376.1 | 21291272 | 5.53606457 | AA | AG | GG | 4.32199546 |
| common_711 | | | | | | | |
| common_5231 | NC_044379.1 | 35666392 | 5.41079957 | CC | CG | GG | 4.31824926 |
| GBScompat_{—} | NC_044371.1 | 23661598 | 5.25929541 | AA | AG | GG | 3.69924812 |
| common_137 | | | | | | | |
| common_2737 | NC_044374.1 | 75450033 | 5.19523943 | AA | AC | CC | 4.20057929 |
| common_1721 | NC_044373.1 | 2378938 | 4.88170154 | AA | AG | GG | 4.45575221 |

| SNP | Mean 2 | Mean 3 | Var | Count 1 | Count 2 | Count 3 |
|---|---|---|---|---|---|---|
| common_1631 | 4.34259259 | 4.22316684 | 0.3 | 425 | 972 | 941 |
| common_1626 | 3.94369369 | 4.3534855 | 0.4 | 273 | 444 | 1621 |
| common_4470 | 4.35017422 | 4.22471191 | 0.2 | 202 | 574 | 1562 |
| common_946 | 4.61568627 | 5.2244898 | 1.2 | 1475 | 765 | 98 |
| common_3557 | 4.41245791 | 4.43103448 | 0.3 | 1454 | 594 | 290 |
| common_3426 | 4.17523057 | 3.63487738 | 0.8 | 1212 | 759 | 367 |
| common_299 | 4.34693878 | 4.5754717 | 0.4 | 1889 | 343 | 106 |
| common_5100 | 4.28456592 | 4.42276423 | 0.2 | 1347 | 622 | 369 |
| common_2434 | 4.27941176 | 4.51388889 | 0.3 | 2198 | 68 | 72 |
| common_941 | 4.30410959 | 2.84711779 | 1.8 | 1209 | 730 | 399 |
| common_5391 | 4.18380744 | 4.39134126 | 0.3 | 703 | 457 | 1178 |
| common_2630 | 4.07423581 | 4.49924357 | 0.9 | 329 | 687 | 1322 |
| common_2031 | 4.39473684 | 3.74447174 | 0.7 | 1209 | 722 | 407 |
| GBScompat_{—} | 4.10596026 | 4 | 0.3 | 2035 | 302 | 1 |
| common_412 | | | | | | |
| common_4191 | 4.20081967 | 4.21428571 | 0.1 | 2024 | 244 | 70 |
| common_5139 | 4.1988743 | 4.35337124 | 0.3 | 574 | 533 | 1231 |
| common_679 | 3.79057592 | 2.79365079 | 1.5 | 2084 | 191 | 63 |
| GBScompat_{—} | 3.95300261 | 4.2998679 | 0.4 | 441 | 383 | 1514 |
| common_711 | | | | | | |
| common_5231 | 4.18670438 | 4.06007067 | 0.3 | 1348 | 707 | 283 |
| GBScompat_{—} | 4.19770408 | 4.39052795 | 0.7 | 266 | 784 | 1288 |
| common_137 | | | | | | |
| common_2737 | 4.3255814 | 4.28624535 | 0.1 | 1381 | 688 | 269 |
| common_1721 | 4.19917441 | 4.19520174 | 0.3 | 452 | 969 | 917 |

TABLE 3
A QTL list is given associating each SNP with an assigned QTL.

| QTL | Chromosome | SNPs | Position |
|---|---|---|---|
| QTL1 | NC_044370.1 | common_299 | 32633325 |
| QTL2 | NC_044371.1 | common_679 | 13434356 |
| QTL3 | NC_044371.1 | GBScompat_{—} | 23661598 |
| | | common_137 | |
| QTL4 | NC_044371.1 | common_941, | 77104557 to 78310503 |
| | | common_946 | |
| QTL5 | NC_044372.1 | common_1626, | 83254930 to 83988588 |
| | | common_1631 | |
| QTL6 | NC_044373.1 | common_1721 | 2378938 |
| QTL7 | NC_044373.1 | common_2031 | 44346866 |
| QTL8 | NC_044373.1 | GBScompat_{—} | 60699429 |
| | | common_412 | |
| QTL9 | NC_044374.1 | common_2434 | 4747237 |
| QTL10 | NC_044374.1 | common_2630 | 52581732 |
| QTL11 | NC_044374.1 | common_2737 | 75450033 |
| QTL12 | NC_044375.1 | common_3426 | 91437428 |
| QTL13 | NC_044376.1 | common_3557 | 7991454 |
| QTL14 | NC_044376.1 | GBScompat_{—} | 21291272 |
| | | common_711 | |
| QTL15 | NC_044377.1 | common_4191 | 29886295 |
| QTL16 | NC_044377.1 | common_4470 | 71514027 |
| QTL17 | NC_044379.1 | common 5100 | 15575449 |
| QTL18 | NC_044379.1 | common_5139 | 25700349 |
| QTL19 | NC_044379.1 | common_5231 | 35666392 |
| QTL20 | NC_044379.1 | common_5391 | 52728955 |

TABLE 4
Detailed information of each of the SNPs associated with flowering time in Cannabis
as provided in Table 2. The “ref” reference allele based on the CS10 genome, and the
identified “alt” alternative allele based on the SNP marker panel are given for
each SNP. The “context sequence” is given with the SNP given in brackets. All of
the sequences and alleles are provided with reference to the plus strand.

| SNP | Ref | Alt | Context sequence |
|---|---|---|---|
| common_1631 | T | C | CCATTACAAAACTTGAATTATATAAAGAAAAATGTGAATCAATAAAATTAC |
| | | | AAAAGAGAGAAAAAAAAAAGTATATAAGAGAAAGAAAAACCATATCCTCC |
| | | | TCAAAAGGGTATGTATATTTTTCTGCAAATTTGACATTGTAACAACTACTC |
| | | | TTGATTTTCTCAATATATTTTTAATTTTTTTTTTATCTTTGACTATTAT[T/ |
| | | | C]AATTGCAGTAGTTAGAGGTGGAATAGCATTGTTTGGTGTTGATTGAATT |
| | | | GGAAAATTAAGACGCCAAAGCATGATCTTAGAGCACCCATCTGGTGGGAT |
| | | | ACCTTTTATTTCAGTTTCATCTTCAAGAACCACTTGCTTCACAAATTTGAT |
| | | | TGAATCCTGCTCAAAAATATATTAATTTAATAATAATTAAGTACAGCTAAT |
| | | | (SEQ ID NO: 1) |
| common_1626 | C | T | CTAGTACAACGATTCAGCTAAAAACTCAGGAAACTCATCAAGCAACATCT |
| | | | CGTTCTCTCGAGCTAGCAAGGCTTCCACAAATGAAGTATTATCCATATCT |
| | | | TTAGATCCTTCACTCTGATTTTGGCTTAGATCATTCTGTGAAGAAGAATA |
| | | | ATTCATCAACTCAGCCATTGATGTCTCATGTTCTTGAGCTTGATCATTTG |
| | | | G[C/T]AAAAATGCATCTTCAAGAAGAAAGTCATTCCAATTGAAGCTGGCT |
| | | | ACACCAGCTAGAGGTGGCATCTCTTGGGTACTGGATGAAGATGATGATG |
| | | | AGGGTGAAGAAAATGTTGCTTCTGGGTAGAAACAAGGCAATAATGAAGA |
| | | | TTCATAATTATTGGGTTTGTAGTTTGAAGCTTCAGTACTGACCAACTTTAT |
| | | | GGCTTGG (SEQ ID NO: 2) |
| common_4470 | A | T | GGAGATGATCAAGAAAGGAAAACATCCTAATGTTGTGACATATGCAATG |
| | | | ATAATGGAAGGTTTGTGTTTGTTGGGAAAGTATTCGGAAGCAAAGAAGA |
| | | | TGATGTTTGATATGGATTATCGAGGGTGTAAACCAAAGCTTGTGAACTTT |
| | | | GGTATTTTGATGACTGATCTTGGAAAAAGAGGTAAGATTGAGGAGGCAA |
| | | | GAGC[A/T]TTAGTTAGTGAGATGAAGAAAAGGAAGTTTAAACCTGATGTT |
| | | | GTGAGCTATAATATATTGATAAATTATCTGTGCAAGGAAGGTAAGGCAAT |
| | | | GGAGGCATATAAAATTTTGATGGAAATGCAAGTTGAAGGTTGTGAACCA |
| | | | AATGCAGCTACATACAGGATGATGGTTGATGGGTTTTGCAGGGTGGGC |
| | | | GATTTCGAAGGT (SEQ ID NO: 3) |
| common_946 | A | G | CGGAAGTAACCACTTCACTAATTGCAATATGGTATACTTAGCTTGATACA |
| | | | TAGTTTTCTTGTTACATCTTAATCCATTCTGATTATTTTTCTGCTGTCTTTG |
| | | | CAACTGATGAATAGTTTCTTGGCTATGTATOTTCAGTGTAAACACAGAAG |
| | | | CCATAACATTCAACTTTACGTATGGCCTTAGTGCTGCAGCCAGGTATGG[ |
| | | | A/G]ATAAATCAAACTTACACTAATCTTTCAAATTCAACATTTCATTTATCTC |
| | | | ATTTTTGGTATTGTTGTTACAACAGCACAAGGGTATCGAATGAGTTAGGG |
| | | | GCAGGGCGTCCGGACAGAGCTAAGAGTGCAATGATTGTTACCCTGAAG |
| | | | CTATGTGGAGTTCTTGCCTTGATACTAGTTTTGGCTCTAGGATTCGGACA |
| | | | CAAC (SEQ ID NO: 4) |
| common_3557 | C | A | TTTTATCCAATCCAATCCTATATCAAACCCCACTACCCAAACGTAACCAA |
| | | | CGTCCACCCAAAAAAAAAAGCAAATGGAGCTGCAATCCGAACAGGTATA |
| | | | ATATCATAATGCTAGCTCGGTTAGTGAAAAACACCACATTTTAAAAGAAC |
| | | | TGCATGTATTTCAAAAGCAGTATTACCTTCCAACTGTAGGAAACATTTTT |
| | | | CC[C/A]GCTGGCTGAAGAAACCGATCTCTTGCAATTACATAGGACTCCAG |
| | | | CATTCTTTCATTGACTAATAAGGTTCCTAAAGACACAAAGAGAAATTATAT |
| | | | ATGAACTCACTCCAGCAAAAATAGAGCTTACAATACCCATAACCCACTTT |
| | | | AAATCAACACTGTTGATAATTTTTTATTCTAGTTTCCTTTTTCTGGTTTTCC |
| | | | TTAT (SEQ ID NO: 5) |
| common_3426 | A | C | AAACGTTACCAAACCAGAAGAGACCAACATTGCTACATTCTCGATTACCA |
| | | | GTGCTACAAACCAACCGACGATCGAAAGCTCAACACCGAGTTTTGTGGC |
| | | | GAACTCATTAGGCGGACCAAGAATCTAGGCCTCATCGAGTACAAGTTTC |
| | | | TCCTCAAGGCTATTGTCAGTGCTGGAATCGGTGAACAGACTTACGCTCC |
| | | | GAGA[A/C]TCATCTTCGACGGTCGTGAAGATTCTCCTACTATTGCTGATG |
| | | | GAATCTCTGAGATGGAGGAGTTTTTCTTTGACAGCGTTGGAAGACTTCT |
| | | | CAGACGTAACGGAATATCTCCGTCTCAGATCGATGTTCTCGTTGTTAAC |
| | | | GTTTCGATGCTTTCGACTGTTCCTTCTTTGTCTTCTCGGATTATAAATCAT |
| | | | TACAAGATGA (SEQ ID NO: 6) |
| common_299 | T | C | AAAAAATATGGATGAAAAATAAAATAAACATCACAGCTTTTTGTACCCCC |
| | | | TCTCTCTCTAAAGACGTACGTACTAATAATAATAATAATTAACTACAAAGT |
| | | | TTGGATAATTAAAACAAGTTGTTTTATAAGTTTTGATGATGAGAAGATAGT |
| | | | ATATATAATATATTGGATCATATCAAATCAACTAAACAGCTAGCTGTTG[T/ |
| | | | C]TGTGTTTGATGAGATGATTGATCTTGATCTTCATTGAAGCACAAGTGA |
| | | | AGATTTAGTGATTTTCCAGAAGTAATAAGAGAAGAAGTAGTTAGTTGTAG |
| | | | TGGTGGTTGCATTAGAAGCATTTCTCTTTGGAGATAACAACAATAGGTTT |
| | | | GGAATGTGGTTGGAGTCACATTCAACTGAAACCCTACCCCAAATAGGAA |
| | | | ATCC (SEQ ID NO: 7 |
| common_5100 | T | C | TCATCATCATCATCATAACCCTCATCATCACCCTCCAACCTCTCTGAATC |
| | | | AGATCAATCTACCACCCTGCACACCTCAAGAATTTCATGGTTCTTTTCTC |
| | | | TGTTTCACTTATTGCCTCTTCTCTGTCTTCTTTCATTCTTTTTTTAGATTAT |
| | | | TGAGATACTATAATTGTTTCATGGTGGCAGGAGTGGCATCGTTTCTAGG[ |
| | | | T/C]AAGAGATCGGTATCGTTTTCGGGTATCGAGCTAGGAGAAGAAGGCA |
| | | | ATGGAGGAGAAGATGATTTATCCGATGATGGATCTCAAGCAGGGGAAAA |
| | | | GAAGAGGAGACTTAATATGGAACAGGTTAAGACCCTTGAGAAGAACTTT |
| | | | GAATTGGGGAACAAGCTTGAGCCAGAAAGGAAAATGCAGCTAGCTAGA |
| | | | GCTCTTGGT (SEQ ID NO: 8) |
| common_2434 | G | A | TATATTGTTTTGGTTACAGTTGGAATTGAGGCAGAGGATGAGAAGGTTA |
| | | | ACTTCCTTTTGACTGAGGTCAAGGGCAAAGATCTCACTGAGCTAATTGC |
| | | | TTCCGGAAGGGAGAAGCTAGCATCAGTTCCGTCAGGTGGTGGTGGTGG |
| | | | TGCAGTTGCTTACTCTGCACCATCAGGTGGAGCAGGCGCCGCCCCAGC |
| | | | TGCTGCT[G/A]CCGAGTCAAAGAAGGAAGAGAAAGTAGAAGAGAAAGAA |
| | | | GAGTCAGATGATGTAAGTTTCATATAGTTGTTAAGTCTTTAAAGTCTCTG |
| | | | GTTTTGGCTGTTTTTAAGTCATTGAATTTGCTCTAATGGTATGCTGTTTAT |
| | | | TCTTTGTTTCAGGATATGGGTTTCAGTCTCTTCGACTAAAAATCTTTAGC |
| | | | TTTTATCAGGAA (SEQ ID NO: 9) |
| common_941 | G | A | GTGAGGGATTTTTCCATTGAGACCCTGAATTTAGTGTAGTGCTCGTACA |
| | | | AACCACCATCCAATGCCACTACAGACTTCTGTTTATCTCCCAATTTTACT |
| | | | GTGTCTCTTCCCAATTTCTTGAGGATTCCTAAGATCCCTGCGGCTGATA |
| | | | GTCGAGCTCCTCGAGTGGCGACAATATCACAAATCTCCACTATTGTTTTT |
| | | | CTC[G/A]TTTTGAGGGAGGTGTTAGATATCTGACAAAGAGAGATGGACAA |
| | | | CTTATTATAACCATCAAACGTGGAAATAAACTGGTACTAATTAAGGAAAA |
| | | | TAATATATTCTATGATGTTTAGTAGGGTCCTAATGTTTAGTTCAACTGCA |
| | | | GAGAAAGTAATCAACTCAAAGGTTGAACTGTCCCTAACTAGTTAGTCTTT |
| | | | CCTCACCT (SEQ ID NO: 10) |
| common_5391 | G | T | GTTCGATGAAGTCAATACCCGAACCCCTATAATCCTTGCAAAACTCGAA |
| | | | ATTTGAAATGCTAAGAAAGGATCATGTAAGGTGCGTTCTTGACTTCTCCA |
| | | | AAATATTTGCATTTCCTCAACACCTTAGATTTCTTTATCTTACTTCTTTTTC |
| | | | TTTCCCCTGCTGCTTTTCACTAATTTCGACCATCTTCTTCTATCAAATATI |
| | | | G/T]ACTGCAAAAAACACTAATCAAAACCCCAATTAATCACAACTGAGAAG |
| | | | GCAATCGGATTATTGCATTGTTATAACTAATTATAGTACTACATTACACTT |
| | | | GCACTATATATTTTGTGCTCCAAAACTCATGAATGAAATAATCACGTGCC |
| | | | ATTGTTTTTGGCAATATATTGAGTTTGTAAGAATTATAGAAAATCTCTATT |
| | | | AA (SEQ ID NO: 11) |
| common_2630 | A | G | TGGATCTCATTAAAGTGACTAGATATTTGATACTAATGGTCAAGTTCACT |
| | | | CTTGACTTGTACATATTTCCATGATCAAAACTACCCTAATAAAGTTGCTC |
| | | | ATTGTGACTTATGAGGTGTGCTAGCGTCATTGCAGACTCTTTAATCATAG |
| | | | ATTGCACTTATATATAAAGTTGCTTTAAATAAAGAGTCGTGAGCTTCAAC |
| | | | T[A/G]AAAACCTCAAAGATATCTCATTTTATCATTTATTCTTTOTTAGAATT |
| | | | TTTTAACACCCTTTGAACTATATATAATGGAAGTTGAGAGGACTCCAGCT |
| | | | ACTAAAGAAAGCAAATATGAGGTAAGTTTCACCAAGAAACATATTGTGAA |
| | | | AGCATTAAATATTTCCTCATTACCAAAATCAAGCATCTTAACCCTTTCCAA |
| | | | TTT (SEQ ID NO: 12) |
| common_2031 | C | T | CACAAACTCGGCCACAAATTCAAGTAGTTCTGGCTTAAACATCTGATTCC |
| | | | AAAGTACCAAAGCCCACTCACTTGGCTGGTTAAGCCCATAGGCTTCAGC |
| | | | AACTATTAGTGCCTCTTGAAAACGGGACTGCTCAACTAAGACTCTCCTG |
| | | | GCATTTGTCTCTGACAGGTAAAGCCATTGAATGTCAGGCATCCGGATTT |
| | | | GGAG[C/T]GACAGAAGGGAAGCCTGAGCACAAGCTCTACGTGCTTTGTT |
| | | | GCCAGCATCAAGGGAGGAGTGAACTTCAGCAGCTTCAATGAAATAGCG |
| | | | CATAGCATCTAATAGGTCTTCATTCTGGTCATTGTCTTTACGACCAAACC |
| | | | ACTGCCCAGAGGACTGATCTGCTCGAGATTCTAAAAGAGCAGCTGTTTC |
| | | | ATGTTTCATGTCA (SEQ ID NO: 13) |
| GBScompat | A | G | GCCCATTCATACCTTTATGGTTGTGGCTGCATAGTCCACACAGCCATAT |
| common_412 | | | GTATTGCAGTCCACTGGAGCAACATTTAAGAACATTCTCAGAGTTTTAAC |
| | | | ACATAACTTCCAACAATAAACTAGCATTCCTGTGACACTTGTTACTTATCT |
| | | | AAGTTTGCTATAACAAGATTATGTGCTACATTTTGGTAATGTTTAAGCATC |
| | | | [A/G]AATTGATCCAATTCGAAAAACTTGAAGTAGATGGAAAATTGATGCTA |
| | | | CATTGGTAGCTGCTAGTTATTATCACAAAGATTAATATTTGGAATATGTAA |
| | | | ATGTACGTATGAACAATGAACTGTAATTTCAGTCTTCTTATATTTAACAAG |
| | | | AAGTAATCAAGTTAGGGTATTCCAAAAACAGTGTTAAAATTGAGTTTCTT |
| | | | TG (SEQ ID NO: 14) |
| common_4191 | T | G | TCCCGTTACGGCCCAGCAGCCACCGTCTTCAACGGCCCAGTTAGGAAG |
| | | | TGGAAGAAGAAATGGGTCCATGTTTCCTCCTCCTCTTCTTCTTCAACCCT |
| | | | CAACGCCTACAACCACAACCCTCAATCACAATCCAACGGCACCCCAACC |
| | | | CCCCGCCTCCTCCTCTGCCGATGGACTCCCGCCACCGCTACTTCAGCC |
| | | | GCCGCT[T/G]CCGACGGCTCCGGCGGAACGCAGTTGGAAGAGCCACCG |
| | | | AGGAGGAAGTTCCGGTATACTCCTGTAAGTGAAAATCACTTGATTATGC |
| | | | TTTAGGGTTTATGGGTATTATTGGTTCTGCTGTTACATATATGGGTTTGT |
| | | | TGGCGTGTCTGTGTTTGGTTAGATTATGCATATGAATATGAAATATGGAT |
| | | | TTGTTTTCTTTTCT (SEQ ID NO: 15) |
| common_5139 | C | A | AAGTTAACAGGTATGTTTCAGATTTTGTCTTACTTTCAGCATTTTCTTCGC |
| | | | TTTTTCAAGCTCAAAAGGATCAGGGTGATTTGCACTGAAAACAGTCTCCA |
| | | | CCTGAAAAATAAATGAGTGTAACTAAGTAAACAGAAAACAACATACCAAC |
| | | | AAATTTGGCTGAAATACATTTATCATACATACCTCCTTGACCAATGCATC[ |
| | | | C/A]GTGTTAAGTAATTCAATATCATCTGATACTTTTTTTGCAATCCCATTT |
| | | | TGTGTTGGGGGAAAATCTTTCTTTGGCTGCACCTTACTTGGTCCTCTCC |
| | | | CTCTTCCAGAACCTGGAATGGCACCACCACGATTCGAGGGTTTCTTGTG |
| | | | GCCACGACCATGGCCACCGTGGTTTCCTCCATGCGATATAACCAAATCA |
| | | | GCACCA (SEQ ID NO: 16) |
| common_679 | A | G | AACTTAAAAGGAAAAGAATTGAACTTGAAACTCTGTATAGCATCATGGCT |
| | | | GTTTTCTTTGTCTTCTCTTCTCTTTGAATTCAATAGGTACTTGATCTGTGA |
| | | | GAAGCTGGCACTGTTTTTATTGGTTCATACCAATTTTAAGATATTCCTTTT |
| | | | TATTTTATTTTTTGGTTTACGTAATATTTAATTTGAACATGTAACAGAA[A/G |
| | | | ITTGGGCTTTGCATCCTGTAAGCGGAACTTGGAAGCTACAGAAGGAATTA |
| | | | ATGTTCTTATTGAGAAGAAAAATAATGATGGGAAGTGGGCCTATGGTTTA |
| | | | TCATGCATTGAATATACTGAGTTTGAAAAGTTTGGGATTGCAGATGGACA |
| | | | TCATTCAACCAATAGGTATAGTCTGGTTCACTTACAAAGTTGGCATAATC |
| | | | AA (SEQ ID NO: 17) |
| GBScompat | C | T | ATTTAACTCTCCGGTTATGTCGACAAAGCCCCGAGCTGTTCTTGACAGC |
| common_711 | | | GCTAGCAAAATGCGAGCGTCTTATGGATTGAAGCAGGGGCAGTCCCGT |
| | | | CTTTTTCACGAGCTCCCATCTGGGCTGAATATGGAGTTGATTGTACAGA |
| | | | AGGGTGCTGTAGATAATAAAGACCCAGATGATAAATGCCAGACAAGAAT |
| | | | TGATAA[C/T]CCATCTCTGGTTTTTGTTCATGGAAGCTATCATGCTGCTTG |
| | | | GTGCTGGGCTGAACACTGGTTACCCTTCTTTTCATCACATGGCTACGAT |
| | | | TGCTATGCTCTTAGCTTGTTGGGCCAGGTTCTTTCTCTCATCTTACCTTC |
| | | | TCCTTTTCATCAAGGCTGTCAAATCCGTTTCTTTTCATCTTTAATGAATCG |
| | | | TTAAATGATT (SEQ ID NO: 18) |
| common_5231 | C | G | GGCAAAGCAAAAGCAAATTAAGACAAACCCACATATCACAATTCACAACT |
| | | | CAAAACTCAAAACTCACAAGTTATTATATATTACTCCATTCCTTTGTTTCT |
| | | | GAGAACCATTAGAGAAAAGAATGGGTGCCGTTGTGTTAAACCAGAGTGT |
| | | | ACACATAAATCCCAGCAAAGGGCCTCAAAAATTAATTAACAACGATGTGA |
| | | | T[C/G]GAAGAAATTGGAGGGCTTATTAAAGTGTACAAAGACGGAAAAGTC |
| | | | GAAAGACCAGAGGTAGTGCCATGTGTCACTGCTTCATTGGCTGATCACG |
| | | | ATCAACTGGGATCAGCAGCAGTGATTTCTACGGACGTGGTCATTGACAA |
| | | | GTCCACCAATGTTTGGGCTCGTTTTTATGTTCCAATTTTGGCTCAGAAAG |
| | | | ACAAAATC (SEQ ID NO: 19) |
| GBScompat | G | A | TGCAGCTCACTGCCTTAAACCGCATCTTTTCAACATATAGATCTGCAGAA |
| common_137 | | | ATTTTAATACTGTGTTATATAATATGATCATTCTTTTAAAAAAAAAATCATT |
| | | | AAAATGGAGATAGCAACTTGGCTAAAACTCACCCATTACACAAGTGTTCA |
| | | | AATTTGGAGCTGTTTTAAAGAGCCTGAAGAACATAACATAGTTTCCAGA[ |
| | | | G/A]GTAACAGCAGCACGAACTGCAAGAGCATGCTTTACAGCATTATCCC |
| | | | TTTTTGCTTCCCTTGATAATCTGAACTCAATTTCACAACAATATTGAAGGT |
| | | | CATTACAAGAGTTTCAACATAGTAGAAGGATGGTAAATAGCTAATGAAAT |
| | | | TTCCTCAAATCATAACTGACCTTGACATGGATGATACAAGATCTCTGTTG |
| | | | TTAC (SEQ ID NO: 20) |
| common_2737 | C | A | TGTTACTGATTCTCTCATAAGATAGAGAAGAGAAAAAAAAAAAAGGGGA |
| | | | GGAGGGAAGATTTTCAGAATATATGTTCTTCCCTTACTTTGCATCAAACC |
| | | | CAGCTTTTATTATATATATGATTAATAATATATATATTTATATATATTTCTTC |
| | | | TGTCCATATTAATTTTCTTCCTTAAGCCCATCCAAAACCAAATCCTCTA[C/ |
| | | | A]TGAAGCTTATATTCCCTTTTGTCAGCAAGAGTCCATTTATCTTGACCCA |
| | | | CGTTTCAAGCATTAAATTTTGTGAGAATACAAGTTTTGATACCATTCCCT |
| | | | GTCCAAAGGGTACTCTCTAGCTAATAATCACTATATATCCAAATTAATTC |
| | | | AAACCATATTCACATTAAGAAATAACCAAAGTTAGCTCTCTCTTTCTCTCA |
| | | | T (SEQ ID NO: 21) |
| common_1721 | A | G | AGATGATAACTGTTGTGGTGGACAACCAGACGGTGGTATGGATGAACTT |
| | | | TTAGCTGTTTTGGGTTATAAAGTTAGGTCATCTGACATGGCTGAAGTTGC |
| | | | TCAAAAGCTTGAACAGCTTGAAGAAGCTATGTGTAGTGTTCAACAAGATA |
| | | | ATCTTTCACAACTTGCTTCTGATACTGTTCATTATAATCCTTCTGATTTAT |
| | | | C[A/G]ACATGGTTGGAAAGTATGCTTACAGAGCTTAATCCTTCTCCTCCT |
| | | | AATTTTGATTCTGTAATGGTACCACCACCACCACCACCACCACAATCACA |
| | | | ACCTCAAAGGCCTTCGATTATTGAAGAAACTTCTTACTTAGCTCCAGCTG |
| | | | AATCTTCAACCATAACTTCTATTGATTTCCCAGATCAGAGAAATCAAAAC |
| | | | TCGTTA (SEQ ID NO: 22) |

TABLE 5
Targeted sequencing primers (5′ to 3′) for the SNPs identified in Table 2,
as described in Example 1.

| | Forward | Reverse | Forward | Reverse | Forward | Reverse |
|---|---|---|---|---|---|---|
| SNP | Primer 1 | Primer 1 | Primer 2 | Primer 2 | Primer 3 | Primer 3 |
| common_1631 | ACCAT | TCAAA | ACCAT | TCAAA | AACCA | TCAAA |
| | ATCCT | TTTGT | ATCCT | TTTGT | TATCC | TTTGT |
| | CCTCA | GAAGC | CCTCA | GAAGC | TCCTC | GAAGC |
| | AAAGG | AAGTG | AAAGG | AAGTG | AAAAG | AAGTG |
| | GT | GTT | GT | GT | GGT | GT |
| | (SEQ ID | (SEQ ID | (SEQ ID | (SEQ ID | (SEQ ID | (SEQ ID |
| | NO: 23) | NO: 24) | NO: 23) | NO: 25) | NO: 26) | NO: 25) |
| common_1626 | TCTCT | GCCTT | TTCTC | GCCTT | GAGCT | GCCTT |
| | CGAGC | GTTTC | TCGAG | GTTTC | AGCAA | GTTTC |
| | TAGCA | TACCC | CTAGC | TACCC | GGCTT | TACCC |
| | AGGCT | AGAAG | AAGGC | AGAAG | CCACA | AGAAG |
| | (SEQ ID | C | (SEQ ID | C | (SEQ ID | C |
| | NO: 27) | (SEQ ID | NO: 29) | (SEQ ID | NO: 30) | (SEQ ID |
| | | NO: 28) | | NO: 28) | | NO: 28) |
| common_4470 | TCGAG | CTTCG | TCGAG | ACCTT | TCGAG | CCCTG |
| | GGTGT | AAATC | GGTGT | CGAAA | GGTGT | CAAAA |
| | AAACC | GCCCA | AAACC | TCGCC | AAACC | CCCAT |
| | AAAGC | CCCT | AAAGC | CACC | AAAGC | CAACC |
| | T | (SEQ ID | T | (SEQ ID | T | (SEQ ID |
| | (SEQ ID | NO: 32) | (SEQ ID | NO: 33) | (SEQ ID | NO: 34) |
| | NO: 31) | | NO: 31) | | NO: 31) | |
| common_946 | ACGTA | TGTGT | CGTAT | TGTGT | ACGTA | GTGTC |
| | TGGCC | CCGAA | GGCCT | CCGAA | TGGCC | CGAAT |
| | TTAGT | TCCTA | TAGTG | TCCTA | TTAGT | CCTAG |
| | GCTGC | GAGCC | CTGCA | GAGCC | GCTGC | AGCCA |
| | (SEQ ID | (SEQ ID | (SEQ ID | (SEQ ID | (SEQ ID | (SEQ ID |
| | NO: 35) | NO: 36) | NO: 37) | NO: 36) | NO: 35) | NO: 38) |
| common_3557 | AAACC | GCTCT | ACCCA | GCTCT | AAACC | GCTCT |
| | CCACT | ATTTT | AACGT | ATTTT | CCACT | ATTTT |
| | ACCCA | TGCTG | AAACC | TGCTG | ACCCA | TGCTG |
| | AACGT | GAGTG | CCACT | GAGTG | AACGT | GAGTG |
| | (SEQ ID | AGTT | (SEQ ID | AG | (SEQ ID | AGT |
| | NO: 39) | (SEQ ID | NO: 39) | (SEQ ID | NO: 39) | (SEQ ID |
| | | NO: 40) | | NO: 41) | | NO: 42) |
| common_3426 | GTGCT | ACATC | TGCTA | ACATC | TCGAA | AGAAG |
| | ACAAA | GATCT | CAAAC | GATCT | AGCTC | GAACA |
| | CCAAC | GAGAC | CAACC | GAGAC | AACAC | GTCGA |
| | CGACG | GGAGA | GACGA | GGAGA | CGAGT | AAGCA |
| | (SEQ ID | (SEQ ID | (SEQ ID | (SEQ ID | (SEQ ID | (SEQ ID |
| | NO: 43) | NO: 44) | NO: 45) | NO: 44) | NO: 46) | NO: 47) |
| common_299 | ACAGC | TGCTT | TCACA | GCTTC | TCACA | TGCTT |
| | TTTTT | CTAAT | GCTTT | TAATG | GCTTT | CTAAT |
| | GTACC | GCAAC | TTGTA | CAACC | TTGTA | GCAAC |
| | CCCTC | CACCA | CCCCC | ACCAC | CCCCC | CACCA |
| | T | C | T | T | T | C |
| | (SEQ ID | (SEQ ID | (SEQ ID | (SEQ ID | (SEQ ID | (SEQ ID |
| | NO: 48) | NO: 49) | NO: 50) | NO: 51) | NO: 50) | NO: 49) |
| common_5100 | ACCCT | CCCCT | ACCCT | TCTCC | ACCCT | CTCCT |
| | CATCA | GCTTG | CATCA | TCTTC | CATCA | CTTCT |
| | TCACC | AGATC | TCACC | TTTTC | TCACC | TTTCC |
| | CTCCA | CATCA | CTCCA | CCCTG | CTCCA | CCTGC |
| | (SEQ ID | (SEQ ID | (SEQ ID | C | (SEQ ID | T |
| | NO: 52) | NO: 53) | NO: 52) | (SEQ ID | NO: 52) | (SEQ ID |
| | | | | NO: 54) | | NO: 55) |
| common_2434 | CGGAA | GTCGA | GCTAA | GTCGA | AGCAT | GTCGA |
| | GGGAG | AGAGA | TTGCT | AGAGA | CAGTT | AGAGA |
| | AAGCT | CTGAA | TCCGG | CTGAA | CCGTC | CTGAA |
| | AGCAT | ACCCA | AAGGG | ACCCA | AGGTG | ACCCA |
| | (SEQ ID | (SEQ ID | (SEQ ID | (SEQ ID | (SEQ ID | (SEQ ID |
| | NO: 56) | NO: 57) | NO: 58) | NO: 57) | NO: 59) | NO: 57) |
| common_941 | GATCC | AGGGA | TTCCT | AGGGA | ATTCC | AGGGA |
| | CTGCG | CAGTT | AAGAT | CAGTT | TAAGA | CAGTT |
| | GCTGA | CAACC | CCCTG | CAACC | TCCCT | CAACC |
| | TAGTC | TTTGA | CGGCT | TTTGA | GCGGC | TTTGA |
| | (SEQ ID | GT | (SEQ ID | GT | (SEQ ID | GT |
| | NO: 60) | (SEQ ID | NO: 62) | (SEQ ID | NO: 63) | (SEQ ID |
| | | NO: 61) | | NO: 61) | | NO: 61) |
| common_5391 | GGTGC | CCAAA | GGTGC | CCAAA | AGGTG | CCAAA |
| | GTTCT | AACAA | GTTCT | AACAA | CGTTC | AACAA |
| | TGACT | TGGCA | TGACT | TGGCA | TTGAC | TGGCA |
| | TCTCC | CGTGA | TCTCC | CGTGA | TTCTC | CGTGA |
| | (SEQ ID | (SEQ ID | A | (SEQ ID | C | (SEQ ID |
| | NO: 64) | NO: 65) | (SEQ ID | NO: 65) | (SEQ ID | NO: 65) |
| | | | NO: 66) | | NO: 67) | |
| common_2630 | GTGTG | TGGAA | TGTGC | TTGGA | TGTGC | TGGAA |
| | CTAGC | AGGGT | TAGCG | AAGGG | TAGCG | AGGGT |
| | GTCAT | TAAGA | TCATT | TTAAG | TCATT | TAAGA |
| | TGCAG | TGCTT | GCAGA | ATGOT | GCAGA | TGCTT |
| | (SEQ ID | GA | (SEQ ID | TGA | (SEQ ID | GA |
| | NO: 68) | (SEQ ID | NO: 70) | (SEQ ID | NO: 70) | (SEQ ID |
| | | NO: 69) | | NO: 71) | | NO: 69) |
| common_2031 | CCCAC | GATCA | CCCAC | AGATC | CCCAC | CAGAT |
| | TCACT | GTCCT | TCACT | AGTCC | TCACT | CAGTC |
| | TGGCT | CTGGG | TGGCT | TCTGG | TGGCT | CTCTG |
| | GGTTA | CAGTG | GGTTA | GCAGT | GGTTA | GGCAG |
| | (SEQ ID | (SEQ ID | (SEQ ID | (SEQ ID | (SEQ ID | (SEQ ID |
| | NO: 72) | NO: 73) | NO: 72) | NO: 74) | NO: 72) | NO: 75) |
| GBScompat | TGCAT | ACAGT | CTGCA | ACAGT | CTGCA | ACAGT |
| common_412 | AGTCC | TCATT | TAGTC | TCATT | TAGTC | TCATT |
| | ACACA | GTTCA | CACAC | GTTCA | CACAC | GTTCA |
| | GCCAT | TACGT | AGCCA | TACGT | AGCCA | TACGT |
| | (SEQ ID | ACATT | (SEQ ID | ACATT | (SEQ ID | ACATT |
| | NO: 76) | (SEQ ID | NO: 78) | (SEQ ID | NO: 78) | T |
| | | NO: 77) | | NO: 77) | | (SEQ ID |
| | | | | | | NO: 79) |
| common_4191 | GGGTC | CCAAA | GGGTC | CACAG | GGGTC | CAAAC |
| | CATGT | CACAG | CATGT | ACACG | CATGT | ACAGA |
| | TTCCT | ACACG | TTCCT | CCAAC | TTCCT | CACGC |
| | CCTCC | CCAAC | CCTCC | AAACC | CCTCC | CAACA |
| | (SEQ ID | (SEQ ID | (SEQ ID | (SEQ ID | (SEQ ID | (SEQ ID |
| | NO: 80) | NO: 81) | NO: 80) | NO: 82) | NO: 80) | NO: 83) |
| common_5139 | AAGGA | ATGGT | AGGAT | ATGGT | GGATC | ATGGT |
| | TCAGG | CGTGG | CAGGG | CGTGG | AGGGT | CGTGG |
| | GTGAT | CCACA | TGATT | CCACA | GATTT | CCACA |
| | TTGCA | AGAAA | TGCAC | AGAAA | GCACT | AGAAA |
| | CT | (SEQ ID | T | (SEQ ID | G | (SEQ ID |
| | (SEQ ID | NO: 85) | (SEQ ID | NO: 85) | (SEQ ID | NO: 85) |
| | NO: 84) | | NO: 86) | | NO: 87) | |
| common_679 | TCTGT | TGATG | TGTGA | TGCCA | TGTGA | TGCCA |
| | GAGAA | TCCAT | GAAGC | ACTTT | GAAGC | ACTTT |
| | GCTGG | CTGCA | TGGCA | GTAAG | TGGCA | GTAAG |
| | CACTG | ATCCC | CTGTT | TGAAC | CTGTT | TGAAC |
| | (SEQ ID | A | (SEQ ID | CA | (SEQ ID | CAG |
| | NO: 88) | (SEQ ID | NO: 90) | (SEQ ID | NO: 90) | (SEQ ID |
| | | NO: 89) | | NO: 91) | | NO: 92) |
| GBScompat | CCCCG | AACCT | CCCCG | CCTGG | CCCCG | GAACC |
| common_711 | AGCTG | GGCCC | AGCTG | CCCAA | AGCTG | TGGCC |
| | TTCTT | AACAA | TTCTT | CAAGC | TTCTT | CAACA |
| | GACA | GCTAA | GACA | TAAGA | GACA | AGCTA |
| | (SEQ ID | (SEQ ID | (SEQ ID | (SEQ ID | (SEQ ID | (SEQ ID |
| | NO: 93) | NO: 94) | NO: 93) | NO: 95) | NO: 93) | NO: 96) |
| common_5231 | AGAAT | TTGTC | AAGAA | TTGTC | GGGTG | TTGTC |
| | GGGTG | AATGA | TGGGT | AATGA | CCGTT | AATGA |
| | CCGTT | CCACG | GCCGT | CCACG | GTGTT | CCACG |
| | GTGTT | TCCGT | TGTGT | TCCGT | AAACC | TCCGT |
| | (SEQ ID | (SEQ ID | (SEQ ID | (SEQ ID | (SEQ ID | (SEQ ID |
| | NO: 97) | NO: 98) | NO: 99) | NO: 98) | NO: 100) | NO: 98) |
| GBScompat | AGCTC | TGCTG | CAGCT | TGCTG | CAGCT | TCGTG |
| common_137 | ACTGC | TAAAG | CACTG | TAAAG | CACTG | CTGCT |
| | CTTAA | CATGC | CCTTA | CATGC | CCTTA | GTTAC |
| | ACCGC | TCTTG | AACCG | TCTTG | AACCG | CTCTG |
| | (SEQ ID | C | (SEQ ID | C | (SEQ ID | (SEQ ID |
| | NO: 101) | (SEQ ID | NO: 103) | (SEQ ID | NO: 103) | NO: 104) |
| | | NO: 102) | | NO: 102) | | |
| common_2737 | AAAAA | AGAGA | GGGGA | AGAGA | GGGGA | AGAGA |
| | GGGGA | GTACC | GGAGG | GTACC | GGAGG | GTACC |
| | GGAGG | CTTTG | GAAGA | CTTTG | GAAGA | CTTTG |
| | GAAGA | GACAG | TTTTC | GACAG | TTTTC | GACAG |
| | (SEQ ID | G | AG | G | A | G |
| | NO: 105) | (SEQ ID | (SEQ ID | (SEQ ID | (SEQ ID | (SEQ ID |
| | | NO: 106) | NO: 107) | NO: 106) | NO: 108) | NO: 106) |
| common_1721 | AGACG | TCGAA | CAGAC | TCGAA | CAGAC | CGAAG |
| | GTGGT | GGCCT | GGTGG | GGCCT | GGTGG | GCCTT |
| | ATGGA | TTGAG | TATGG | TTGAG | TATGG | TGAGG |
| | TGAAC | GTTGT | ATGAA | GTTGT | ATGAA | TTGTG |
| | T | (SEQ ID | CT | (SEQ ID | CT | (SEQ ID |
| | (SEQ ID | NO: 110) | (SEQ ID | NO: 110) | (SEQ ID | NO: 112) |
| | NO: 109) | | NO: 111) | | NO: 111) | |

### Example 2

### Marker Selection

Quantitative traits like flowering time are highly complex and can be regulated by genes or QTLs across the genome. This poses a challenge in identifying specific markers that contribute most significantly to the trait of interest. In this case the inventors identified 20 QTLs that influence flowering time (Table 3). The QTLs can be used to identify plants in which the presence of an allelic variant associated with early or late flowering, providing an industrially applicable tool for selecting plants with these traits preferentially, for genomic selection, and as part of a molecular marker breeding strategy. However, such approaches are not amenable to such large numbers of QTLs. The inventors took an additional approach to identify the minimum number of QTLs, or SNPs, that contribute the most to the phenotypic variation of flowering time and a combination of two different methods was used.

First, using the values for variation in flowering time a regression analysis was conducted to first model and then predict the phenotype using the allelic status of all significant markers identified by the GWAS. The regression analysis conducted is based on the random forest algorithm (Breiman, 2001) as implemented in the ranger package (v.0.12.1 Wright & Ziegler, 2017) using the allele status as a factor. The subset was defined using the markers with the highest variable importance in the model.

To complement the above-described machine learning approach, the inventors deployed the exhaustive Leaps and Bounds variable selection (Furnival & Wilson, 2000) implemented in the leaps package (v.3.1, Lumley, 2020). Shortly, Leaps and Bounds performs a targeted variable sampling and calculates the model predictability with an increasing number of predictors. The described markers were identified as the main contributors to the model predictability.

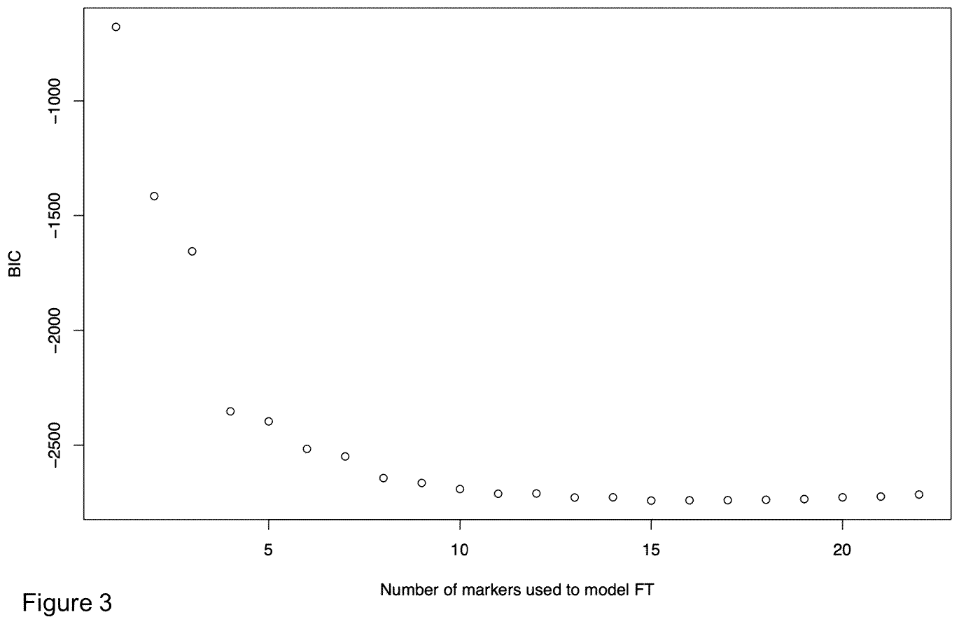

Based on the modeling the inventors were surprised to find that a single SNP, “common_941”, accounted for the largest flowering time variation as compared to the other SNPs identified by GWA. The best fit model that accounted for the most variation with the minimum number of markers was found to be a model with four markers (FIG. 3). The markers that composed this model are “common_941”, “common_2630”, “common_3426”, and “common_679”. These four markers together account for most of the phenotypic variation found in flowering time in the 24 F2 populations used in the present invention that represent both hemp type and HRT-cannabis (FIG. 3). The allelic variation associated with flowering time of these four SNPs can be used as a tool to identify plants with an average propensity for both earlier and later flowering.

1. A method for characterizing a Cannabis spp. plant with respect to a flowering time trait, the method comprising the steps of:
(i) genotyping at least one plant with respect to at least one flowering time QTL by detecting one or more polymorphisms associated with the flowering time trait as defined in Table 2; and
(ii) characterizing the one or more plants with respect to the at least one flowering time QTL as having an early flowering time QTL, a late flowering time QTL or an intermediate flowering time QTL based on the genotype at the polymorphism.

2. The method of claim 1, wherein the polymorphism is selected from the group consisting of “common_941”, “common_2630”, “common_3426”, “common_679”, and combinations thereof, as defined in Table 2.

3. The method of claim 1, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

4. The method of claim 3, wherein the molecular markers are for detecting polymorphisms at regular intervals within the at least one flowering time QTL such that recombination can be excluded, or wherein the molecular markers are for detecting polymorphisms at regular intervals within the at least one flowering time QTL such that recombination can be quantified to estimate linkage disequilibrium between a particular polymorphism and the flowering time phenotype, optionally wherein the molecular markers are designed based on a context sequence for the polymorphism in Table 4 or are selected from the primer pairs as defined in Table 5.

5. (canceled)

6. (canceled)

7. The method of claim 1, wherein the at least one flowering time QTL is selected from one or more QTLs defined in Table 3 with reference to the CS10 reference genome and is defined by one or more polymorphisms associated with the flowering time trait as defined in Table 2, or a genetic marker linked to the QTL.

8. A method of producing a Cannabis spp. plant having a flowering time trait of interest, the method comprising the steps of:
(i) providing a donor parent plant having in its genome at least one flowering time QTL characterized by one or more polymorphisms associated with the flowering time trait of interest as defined Table 2;
(ii) crossing the donor parent plant having the at least one flowering time QTL with at least one recipient parent plant to obtain a progeny population of cannabis plants;
(iii) screening the progeny population of cannabis plants for the presence of the at least one flowering time QTL; and
(iv) selecting one or more progeny plants having the at least one flowering time QTL, wherein the mature plant displays the flowering time trait of interest.

9. The method of claim 8, further comprising:
(v) crossing the one or more progeny plants with the donor recipient plant; or
(vi) selfing the one or more progeny plants.

10. The method of claim 8, wherein the screening comprises genotyping at least one plant from the progeny population with respect to the at least one flowering time QTL by detecting one or more polymorphisms associated with the flowering time trait of interest as defined in Table 2, and optionally wherein the method further comprises a step of genotyping the donor parent plant with respect to the at least one flowering time QTL by detecting one or more polymorphisms associated with the flowering time trait of interest as defined in Table 2, prior to step (i).

11. (canceled)

12. The method of claim 10, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

13. The method of claim 12, wherein the molecular markers are for detecting polymorphisms at regular intervals within the at least one flowering time QTL such that recombination can be excluded or such that recombination can be quantified to estimate linkage disequilibrium between a particular polymorphism and the flowering time trait of interest, optionally wherein the molecular markers are designed based on a context sequence for the polymorphism in Table 4 or are selected from the primer pairs as defined in Table 5.

14. (canceled)

15. The method of claim 8, wherein the at least one flowering time QTL is an early flowering time QTL, a late flowering time QTL, or an intermediate flowering time QTL.

16. The method of claim 8, wherein the polymorphism is selected from the group consisting of “common_941”, “common_2630”, “common_3426”, “common_679”, and combinations thereof, as defined in Table 2.

17. The method of claim 8, wherein the flowering time QTL is selected from one or more QTLs defined in Table 3 with reference to the CS10 reference genome and is defined by one or more polymorphisms associated with the flowering time trait as defined in Table 2, or a genetic marker linked to the QTL.

18. A method of producing a Cannabis spp. plant that has a flowering time trait of interest, the method comprising introducing at least one flowering time QTL characterized by one or more polymorphisms associated with the flowering time trait of interest as defined in Table 2 into a Cannabis spp. plant, wherein said QTL is associated with the flowering time trait of interest in the plant.

19. The method of claim 18, wherein introducing the at least one flowering time QTL comprises crossing a donor parent plant having the at least one flowering time QTL characterized by one or more polymorphisms associated with the flowering time trait of interest with a recipient parent plant.

20. The method of claim 18, wherein introducing the at least one flowering time QTL characterized by one or more polymorphisms associated with flowering time trait of interest comprises genetically modifying the Cannabis spp. plant.

21. The method of claim 18, wherein the flowering time QTL is selected from one or more QTLs defined in Table 3 with reference to the CS10 reference genome and is defined by one or more polymorphisms associated with the flowering time trait as defined in Table 2, or a genetic marker linked to the QTL.

22. (canceled)

23. (canceled)

24. (canceled)

25. A Cannabis spp. plant comprising at least one flowering time QTL characterized by one or more polymorphisms associated with a flowering time trait of interest as defined in Table 2, wherein said flowering time QTL is associated with the flowering time trait of interest in the plant.

26. (canceled)

27. (canceled)
